SEQUENCE LISTING (3 sequences)

SEQ ID NO: 1 - DNA, 19 bp, artificial
  nucleic acid molecule designed to induce RNA interference of Bcl-xL polypeptide expression
aggcgacgagtttgaactg

SEQ ID NO: 2 - DNA, 19 bp, artificial
  nucleic acid molecules designed to induce RNA interference of Bcl-xL polypeptide expression
gtggaagagaacaggactg

SEQ ID NO: 3 - DNA, 19 bp, artificial
  nucleic acid molecules designed to induce RNA interference of Bcl-xL polypeptide expression
ctctgatatgctgtccctg

(19) United States
(12) Patent Application Publication
(10) Pub. No.: US 2024/0165132 A1
(43) Pub. Date: May 23, 2024

(54) METHODS AND MATERIALS FOR TREATING CANCER

(71) Applicant: Mayo Foundation for Medical Education and Research, Rochester, MN (US)

(72) Inventor: Terence C. Burns, Rochester, MN (US)

(21) Appl. No.: 18/281,674

(22) PCT Filed: Apr. 27, 2022
(86) PCT No.: PCT/US2022/026434

§ 371 Date: Sep. 12, 2023

Related U.S. Application Data
(60) Provisional application No. 63/187,726, filed on May 12, 2021.

(51) Int. Cl. A61K 31/635; A61K 31/40; A61K 31/428; A61K 31/4409; A61K 31/451; A61K 31/4545; A61K 31/4725; A61K 31/495; A61K 31/497; A61P 35/00

(52) U.S. Cl. CPC A61K 31/635; A61K 31/40; A61K 31/428; A61K 31/4409; A61K 31/451; A61K 31/4545; A61K 31/4725; A61K 31/495; A61K 31/497; A61P 35/00

(57) ABSTRACT

This document provides methods and materials for treating a mammal (e.g., a human) having cancer (e.g., a central nervous system (CNS) cancer such as a latent CNS cancer). For example, one or more inhibitors of a B-cell lymphoma-extra large (Bcl-xL) polypeptide can be administered to a mammal (e.g., a human) having a CNS cancer to treat the mammal.

### CROSS-REFERENCE TO RELATED APPLICATIONS

This application claims the benefit of U.S. Patent Application Ser. No. 63/187,726, filed on May 12, 2021. The disclosure of the prior application is considered part of (and is incorporated by reference in) the disclosure of this application.

### STATEMENT REGARDING FEDERAL FUNDING

This invention was made with government support under NS109770 awarded by the National Institutes of Health. The government has certain rights in the invention.

### SEQUENCE LISTING

This document includes a Sequence Listing that has been submitted electronically as an ASCII text file named 07039-2038WO1_SequenceListing_ST25.txt. The ASCII text file, created on Apr. 25, 2022, is 2 kilobytes in size. The material in the ASCII text file is hereby incorporated by reference in its entirety.

### TECHNICAL FIELD

This document relates to methods and materials for treating a mammal (e.g., a human) having cancer (e.g., a central nervous system (CNS) cancer such as a latent CNS cancer). For example, one or more inhibitors of a B-cell lymphoma-extra large (Bcl-xL) polypeptide can be administered to a mammal (e.g., a human) having cancer (e.g., a CNS cancer such as a latent CNS cancer) to treat the mammal.

### BACKGROUND INFORMATION

Glioblastoma (GBM) is the most common and deadly malignant brain cancer of the CNS, with a median survival of 12-15 months (see, e.g., Dextraze et al., Oncotarget 8:112992-113001 (2017)). Radiation remains a first line therapy, but tumors invariably recur, typically aggressively, within the prior radiation field (see, e.g., Jeon et al., Tumour Biology, 37:5857-5867 (2016); Kim et al., Cancer Letters, 354:132-141 (2014); Wild-Bode et al., Cancer Res., 61:2744-2750 (2001); and Nizamutdinov et al., World Neurosur., 109:e67-e74 (2018)).

### SUMMARY

This document provides methods and materials for treating mammals (e.g., humans) having cancer (e.g., a CNS cancer such as a latent CNS cancer). In some cases, one or more inhibitors of a Bcl-xL polypeptide can be used to treat mammals (e.g., humans) having cancer (e.g., a CNS cancer such as a latent CNS cancer). For example, one or more inhibitors of a Bcl-xL polypeptide can be administered to a mammal (e.g., a human) having cancer (e.g., a CNS cancer such as a latent CNS cancer) to treat the mammal.

As demonstrated herein, senescent glioma cells are selectively dependent upon Bcl-xL and can be killed using Bcl-xL specific inhibitors. Having the ability to target senescent glioma cancer cells as described herein (e.g., by administering one or more inhibitors of a Bcl-xL polypeptide) provides a unique and unrealized opportunity to selectively ablate residual cancer cells during and/or following treatment thereby delaying, or even preventing, development of a recurrent glioma.

In general, one aspect of this document features methods for treating a mammal having a CNS cancer. The methods can include, or consist essentially of, administering an inhibitor of a Bcl-xL polypeptide to a mammal having a CNS cancer; and administering a chemotherapeutic agent to the mammal. The method can include identifying the mammal as having the CNS cancer. The mammal can be a human. The CNS cancer can include a senescent cancer cell. The mammal can have, prior to the administration of the inhibitor of the Bcl-xL polypeptide, been treated for the CNS cancer with a radiation treatment. The mammal can have, prior to the administration of the inhibitor of the Bcl-xL polypeptide, been treated for the CNS cancer with a chemotherapy treatment. The CNS cancer can be a brain stem glioma, a glioblastoma, an astrocytoma, an oligodendroglioma, an oligoastrocytoma, an ependymoma, a medulloblastoma, or a meningioma. The inhibitor of the Bcl-xL polypeptide can be an inhibitor of Bcl-xL polypeptide activity. The inhibitor of Bcl-xL polypeptide activity can be navitoclax, A1331852, A1155463, or WEHI-539. The inhibitor of the Bcl-xL polypeptide can be an inhibitor of Bcl-xL polypeptide expression. The inhibitor of the Bcl-xL polypeptide expression can be a nucleic acid comprising a nucleic acid sequence set forth in any one of SEQ ID NOs:1-3. The chemotherapeutic agent can be temozolomide, selinexor, APO866, AMG-232, RG7388, or GMX1778. The inhibitor of the Bcl-xL polypeptide and the chemotherapeutic agent can be administered concurrently. The inhibitor of the Bcl-xL polypeptide and the chemotherapeutic agent can be administered in a single composition. The inhibitor of the Bcl-xL polypeptide and the chemotherapeutic agent can be administered separately. The inhibitor of the Bcl-xL polypeptide and the chemotherapeutic agent can be administered within from about 0 days to about 120 months of each other. The mammal, prior to the administering the inhibitor of the Bcl-xL polypeptide and said administering said chemotherapeutic agent, can have not been treated for the CNS cancer for at least 1 month. The mammal can be a mammal that is determined to have responded to the previous treatment.

In another aspect, this document features methods for treating a mammal having a recurrent CNS cancer. The methods can include, or consist essentially of, administering an inhibitor of a Bcl-xL polypeptide to a mammal having a recurrent CNS cancer; and administering a chemotherapeutic agent to the mammal. The method can include identifying the mammal as having the recurrent CNS cancer. The mammal can be a human. The CNS cancer can include a senescent cancer cell. The mammal can have, prior to the administration of the inhibitor of the Bcl-xL polypeptide, been treated for the CNS cancer with a radiation treatment. The mammal can have, prior to the administration of the inhibitor of the Bcl-xL polypeptide, been treated for the CNS cancer with a chemotherapy treatment. The CNS cancer can be a brain stem glioma, a glioblastoma, an astrocytoma, an oligodendroglioma, an oligoastrocytoma, an ependymoma, a medulloblastoma, or a meningioma. The inhibitor of the Bcl-xL polypeptide can be an inhibitor of Bcl-xL polypeptide activity. The inhibitor of the Bcl-xL polypeptide activity can be navitoclax, A1331852, A1155463, or WEHI-539. The inhibitor of the Bcl-xL polypeptide can be an inhibitor of Bcl-xL polypeptide expression. The inhibitor of the Bcl-xL polypeptide expression can be a nucleic acid comprising a nucleic acid sequence set forth in any one of SEQ ID NOs:1-3. The chemotherapeutic agent can be temozolomide, selinexor, APO866, AMG-232, RG7388, or GMX1778. The inhibitor of the Bcl-xL polypeptide and the chemotherapeutic agent can be administered concurrently. The inhibitor of the Bcl-xL polypeptide and the chemotherapeutic agent can be administered in a single composition. The inhibitor of the Bcl-xL polypeptide and the chemotherapeutic agent can be administered separately. The inhibitor of the Bcl-xL polypeptide and the chemotherapeutic agent can be administered within from about 0 days to about 120 months of each other.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B depict the response of glioblastoma (GBM) cells (GBM39) to radiation. FIG. 1A contains microscope images of cells after 15 Gy radiation at days 0, 7, and 14 days post radiation demonstrating increased SA-beta galactosidase staining—a marker of senescence. FIG. 1B is a graph of cell confluence over time with varying radiation levels.

FIGS. 2A-2D show confirmation of therapy-associated senescence in GBM39.

FIG. 2A-B shows SA-beta-galactosidase staining of control, and TMZ (100 μM) treated cells demonstrating presence of senescent glioma in treatment group. FIG. 2C is a western blot analysis showing Bcl-xL expression in 0 Gy and 15 Gy radiated GBM39 over 7 days post-radiation. FIG. 2D is a graph of qRT-PCR of GBM39 following 15 Gy radiation in vitro demonstrating increasing expression of senescence-associated transcripts, p21, pro-apoptotic protein Bax, and anti-apoptotic BH3 family member Bcl-xL over 7 days.

FIG. 3 shows that navitoclax and A1331852 preferentially ablate radiated GBM39. Candidate senolytic drugs were evaluated using GBM39. Drug screening was performed 21 days after radiation. Cells were exposed to drugs for four days prior analysis of cell viability via CellTiter-Glo. Grey and black lines denote the dose-response curve for 15 Gy and 0 Gy radiated cells, respectively. Luminescence values are normalized to 0 nM control for each radiation dose. Navitoclax and A1331852 demonstrated lower IC₅₀ in radiated cells, p<0.0001. The data shown are means±SEM (standard error of mean) of three technical replicates; similar results were obtained in GBM39 with 10 or 20 Gy and GBM76 after 10, 15 or 20 Gy.

FIG. 4 shows navitoclax and A1331852 ablate radiated GBM76. Senolytic drug screening in GBM76 cell line with senolytic drugs targeting different anti-apoptotic pathways, piperlongumine, MDM2 inhibitor (AMG-232), onalespib, dasatinib, quercetin, fisetin, navitoclax, A1331852, and saracatinib. Cells were exposed to ten different concentrations of drug for four days. Circle points denotes non radiated control cells, square points denotes 10 Gy, triangle points denotes 15 Gy and inverted triangle points denotes 20 Gy radiated cells. Luminescence values are normalized individually by 0 nM control. All the data are means±SEM of triplicates at each concentration. Navitoclax and A1331852 have shown lower IC₅₀ radiated cells when compared to non-radiated control.

FIG. 5 shows that multiple representative radiated GBM cell lines are selectively vulnerable to Bcl-xL blockade with multiple agents, and that this vulnerability is not replicated with Bcl-2 blockade. GBM39, 76, 10 and 123 were used to evaluate the senolytic activity of BCL-2-family inhibitors, including Bcl-xL-specific inhibitors (A1331852 and A1155463), a selective BCL-2 inhibitor (venetoclax), and a dual inhibitor of both Bcl-xL and BCL-2 (navitoclax). Dose response curves shown for control non-radiated (black) and 15 Gy radiated (grey) cells. Cells were exposed to drug for 4 days, starting 21 days after radiation. 15 Gy-radiated cells demonstrated higher sensitivity than non-radiated cells to A1331852 A1155463, and navitoclax, but not venetoclax. For all groups, luminescence values are normalized individually to 0 nM control. Data shown are means±SEM of three technical replicates at each concentration. Data shown are representative of multiple confirmatory experiments.

FIG. 6 shows additional representative GBM cell lines are selectively sensitive to Bcl-xL inhibition after radiation. In sum, all cell lines tested to date have demonstrated increased sensitivity to Bcl-xL inhibition after prior radiation. For all experiments, luminescence values are normalized to the 0 nM control for that cell line and radiation dose. All data are means±SD of 3 technical replicates at each concentration.

FIGS. 7A-7C shows that senescent GBM is selectively vulnerable to Bcl-xL inhibitors. FIG. 7A is a graph of the relative survival of senescent and proliferating all GBM cell lines tested with 1 μM A1331852 treatment. FIG. 7B is a graph of the comparison of selective vulnerability of p53 wild type and mutant senescence Glioma to Bcl-xL inhibition. FIG. 7C is a graph demonstrating senolytic effect of A1331852 across the primary and recurrent glioma with or without chemoradiation. Each set of points joined by a line represent a GBM subclone line cells isolated from a different unique patient.

FIGS. 8A-8C show GBM vulnerability to Bcl-xL inhibition depends on radiation timing, radiation dose, and duration of inhibitor exposure. FIG. 8A shows sensitivity to Bcl-xL inhibition at 4 (square), 8 (triangle) and 12 (inverted triangle) days after 15 Gy radiation. All cohorts were exposed to drug for the same amount of time exposure time (4 days), and were analyzed on the 5th day after plating. FIG. 8B shows the impact of prior radiation dose on Bcl-xL inhibitor sensitivity. A1331852 treatment was initiated 4 days following variable doses of radiation. FIG. 8C shows the duration of drug exposure impacts GBM39 vulnerability to A1331852, applied 7 days post radiation for 1 hour to 96 hours with equal total culture duration prior to analysis. Luminescence values are normalized individually by 0 nM control. Graphs show means±SEM of technical triplicates at each concentration.

FIGS. 9A-9C show navitoclax and A1331852 in TMZ-treated GBM39. Evaluation of time-dependency of glioblastoma for Bcl-xL inhibitor-mediated ablation. FIGS. 9A-9B show sensitivity to Bcl-xL inhibitors (A1331852 and A1155463) at different time-points following TMZ treatment; square points denote 4 days post TMZ, triangle points denote 8 days post TMZ and inverted triangle points denote 12 days post TMZ treatment. FIG. 9B shows sensitivity to Bcl-xL inhibitors, A1155463 at different time-points following 15 Gy radiation. Square points denote 4 days post radiation, triangle points denote 8 days post radiation and inverted triangle denote 12 days since radiation with 4 days drug exposure to all cohorts. Luminescence values are normalized individually by 0 nM control. All the data are means±SEM of triplicates at each concentration.

FIGS. 10A-10B show previously radiated GBM39 fail to demonstrate sensitivity to Bcl-xL inhibition after re-entering cell cycle. FIG. 10A shows BCL-2 family inhibitor drug screening in 8 Gy GBM39, those restarted cell proliferation after 6 weeks following radiation. Re-proliferation was detected by regular microscopic evaluation, which is evident by cells becoming confluent after having previously demonstrated no changes in confluency until 6 weeks after radiation. FIG. 10B shows increasing number of cells over 9 days after replating to confirm functional escape from senescence. Cells were re-plated with equal density into 12 wells plate (day 0), and cell counts performed at day 1, 3, 5, and 9. Data represented the mean±SD of three technical replicates. Drug screening (FIG. 10A) was performed using the cells harvested at day 5 and 9. Both demonstrated similar results, data are shown from day 9.

FIG. 11 shows TMZ exposure induces selective vulnerability to Bcl-xL inhibitors. GBM76 and GBM39 were treated with TMZ (100 μM) for 7 days followed by 14 days TMZ-free media prior to treatment with BCL-2 family inhibitors as shown. TMZ-treated cells demonstrated selective vulnerability to Bcl-xL inhibitors (A1331852, A1155463, and navitoclax), but not to the BCL-2-specific inhibitor (venetoclax). For all experiments, luminescence values are normalized individually to 0 nM control. All data are means±SEM of 3 technical replicates at each concentration.

FIGS. 12A-12D show radiated GBM is selectively vulnerable to Bcl-xL siRNA knockdown. FIG. 12A shows western blot confirmation of knockdown following control siRNA and three different constructs of siRNA Bcl-xL. FIG. 12B shows relative cell survival following Bcl-xL knockdown illustrating that 15 Gy radiated cells are more dependent than 0 Gy cells upon Bcl-xL for survival. FIG. 12C shows cell viability upon knocking down Bcl-xL, BCL-W and BCL-2 via siRNA in GBM 39, 7 days after 0 Gy, or 15 Gy radiation. Data are presented normalized to scrambled control of each group. Representative data for each of three different constructs of siRNA Bcl-xL are shown. FIG. 12D shows similar knockdown experiments performed for Bcl-xL, BCL-2 and BCL-W. Radiated cells showed decreased survival relative to non-irradiated cells in the Bcl-xL knock down group only (p<0.0001).

FIG. 13 is a graph of TMZ exposure inducing Bcl-xL dependency, regardless of TMZ resistance. * p<0.02, ***p<0.0001

FIGS. 14A-14C show NAMPT inhibition inducing senescence in surviving cells. FIG. 14A shows that non-radiated GBM164 cells exposed to the NAMPT inhibitor GMX1778 (50 nM×10 days) induced a Bcl-xL-dependent state, with reduced A1331852 IC₅₀ (p<0.0001). FIG. 14B-C show increased SA-beta-gal activity after exposure to GMX1778 indicative of senescence.

FIG. 15 is a plot showing GMB39 cells sensitized to navitoclax in a dose-dependent manner when exposed to selinexor. Results for a single pilot experiment are shown. Error bars show SEM of technical replicates (p<0.001).

FIGS. 16A-16B show an analysis of senescence after radiation in glioblastoma. FIG. 16A shows immunocytochemistry staining for laminin B1 in radiated and 15 Gy radiated GBM39 cells showing loss of laminin B1 in radiated cells. FIG. 16B shows qRT-PCR for senescence-associated genes on in 10 Gy X-irradiated GBM39 cells at 0, 7, 14, and 21 day post-radiation. Relative expression of each gene was compared to that of day 0. CDNKIA, IL-6, BCL-2 and Bcl-xL expressions were significantly increased.

FIGS. 17A-17B show an evaluation of time- and dose-dependency of glioblastoma for Bcl-xL inhibitor-mediated ablation. FIG. 17 shows that vulnerability Bcl-XL inhibition is increased if cells were previously exposed to an MDM2 inhibitor. MDM2 inhibitors increase p53 activity, and serve as radiation sensitizers. Cells following radiation with MDM2 inhibition show higher senescence than radiation alone. Cells were treated with the MDM-2 inhibitor AMG-232 (100 nM) or vehicle prior to radiation. Both with and without radiation, AMG-232 pretreated cells showed greater sensitivity to Bcl-xL inhibitor than control cells without radiation. Greatest sensitivity to Bcl-xL inhibition was seen in radiated cells pre-treated with MDM2 inhibitor. FIG. 17B shows MDM2 inhibition augmenting senolytic cell death. AMG232 pretreatment of p53-WT GBM76 prior to 5 Gy radiation enhances susceptibility to Bcl-xL inhibitor-mediated cell death. IC₅₀ of Bcl-xL inhibitors A1331852 and A1155463. Error bars are SEM. Representative data are from individual experiments.

FIGS. 18A-18C show in vivo senescent glioblastoma tumor ablation by convection enhanced drug delivery. FIG. 18A shows post 1 hour CED MRI showing >70% brain gadolinium penetration in brain parenchyma, which was co-injected with drug. FIG. 18B shows an example of bioluminescence imaging in mice. FIG. 18C shows CED with vehicle or senolytic drug performed at 1 month. Decreased tumor bioluminescence by 84.8% (navitoclax) or 93.1% (A1331852), compared to vehicle (39.7%; overall p=0.02); and small surviving group sizes due to multiple mice succumbing to spinal metastases requiring exclusion from analysis.

FIGS. 19A-19B show that radiation of GBM39 cells in vitro induces a senescent-like phenotype. FIG. 19A shows a graph of the relative expression of Bcl-xL, BAX, and p21 transcript after radiation with 15 Gy at 1, 3, or 7 days post radiation. FIG. 19B shows the relative protein level of Bcl-xL after radiation with 15 Gy 1, 3, or 7 days post radiation.

FIG. 20 shows plots of relative luminescence of radiated GBM39 cells treated with senescent cell anti-apoptotic pathways (SCAPS) inhibitors, including navitoclax and A1331852.

FIG. 21 shows a schematic overview of the p53-p21 pathway involved in senescence following radiation-induced DNA breakdown. MDM2 is a negative regulator of this pathway.

FIG. 22 shows p53-mutant cells that resist senolytic augmentation. The graphs depict the response of GBM6 or GBM 123 cells pretreatment with the MDM2 inhibitor AMG232. The inhibitor did not increase the vulnerability to A1331852 or A1155463 following TMZ or radiation in p53-mutant GBM6 or GM123.

FIG. 23 shows augmented senolytic killing of cells after TMZ treatment. Plots show the relative luminescence of cells pretreated with AMG232 or control cells that are exposed to inhibitor (A1331852 or A1155463) at varying concentrations. AMG232 pretreatment of p53-WT GBM76 prior to TMZ enhances susceptibility to Bcl-xL inhibitor-mediated cell death. IC₅₀ of Bcl-xL inhibitors A1331852 and A1155463. Error bars are SEM. Representative data are from individual experiments.

FIGS. 24A-24C: Ionizing Radiation (IR) increases PUMA mRNA and protein expression in human GBM cells. FIG. 24A) qRT-PCR for PUMA using RNA extracted from GBM39 (i), GBM6 (ii) and GBM164 (iii) cells 24 hours after different varying doses of radiation as compared to the sham (0Gy) radiated group. Y axes demonstrate expression relative to the house-keeping gene GAPDH. Individual data points are shown, representing a biological triplicate in 3 independent experiments. FIG. 24B) Western blotting for PUMA using lysates from GBM39 cells treated with different doses of radiation as compared to sham (0Gy) radiated group. GAPDH was used as a loading control. FIG. 24C) Densitometric analysis of the western blot bands demonstrating the amount of PUMA was normalized to GAPDH, the protein loading control. The relative amount of PUMA is compared to non-radiated (0Gy) cells which was set as 1 after normalizing each band to its corresponding GAPDH loading control.

FIGS. 25A-25D: IR also increases BCL-XL mRNA and protein expression in human GBM cells. FIG. 25A) qRT-PCR for BCL2L1 (AKA: BCL-XL) using RNA extracted from GBM39 (i), GBM6 (ii) and GBM164 (iii) cells, 24 hours after being treated with different doses of IR compared to the sham (0Gy) radiated group. Y axes demonstrate expression relative to the house-keeping gene GAPDH. Individual data points are shown representing a biological triplicate in 3 independent experiments. FIG. 25B) Western blotting for BCL-XL using lysates from GBM39 cells, 24 hours after being treated with different doses of radiation compared to sham (0Gy) radiated group. GAPDH was used as a loading control. FIG. 25C) Densitometric analysis of the western blots was performed, and the amount of BCL-XL was normalized to GAPDH, the protein loading control. The relative amount of BCL-XL from the non-radiated cells was set as 1. FIG. 25D) qRT-PCR performed for BCL-2 (D1), BCL-W (D2), and MCL-1 (D3) using RNA extracted from GBM39 cells, 24 hours after being treated with different doses of radiation compared to the sham (0Gy) radiated group. Y axes demonstrate expression relative to the house-keeping gene GAPDH. Individual data points are shown representing a biological triplicate in 3 independent experiments. Ordinary one-way ANOVA statistical analysis is used for all shown experiments, error bars represent SD values and P values are shown for each graph.

FIGS. 26A-26F: BCL-XL interacts with PUMA more than any other BH3-only family member. FIGS. 26A, 26C, and 26E) Western blotting for BCL2L1 (AKA: BCL-XL) using equal amounts of already immune-precipitated (IP'd) protein lysates from non-irradiated GBM39 (FIG. 26A), GBM6 (FIG. 26C) and GBM164 (FIG. 26E) cells. Equal concentrations from proteins BCL-XL, PUMA, BIM, BID, and BIK were IP'd from lysates acquired from the non-irradiated GBM cells as described in the Materials and Methods. FIGS. 26B, 26D, and 26F) Densitometric analysis of the western blots' bands was performed, and the amount of BCL-XL bound to BIM, BID and BIK was compared to the amount of BCL-XL bound to PUMA. The relative amount of BCL-XL bound to PUMA was set as 1.

FIGS. 27A-27H: IR increases the interaction between BCL-XL and PUMA more than other BH3-BCL-XL interactions. FIGS. 27A, 27C, and 27E) Western blotting for BCL2L1 (AKA: BCL-XL) using equal amounts of already IP'd protein lysates from 15Gy irradiated GBM39 (FIG. 27A), GBM6 (FIG. 27C) and GBM164 (FIG. 27E) cells 24 hours after irradiation. Equal concentrations from proteins BCL-XL, PUMA, BIM, BID, and BIK were IP'd from lysates acquired from 15Gy irradiated GBM cells 24 hours after irradiation. FIGS. 27B, 27D, and 27F) Densitometric analysis of the western blots' bands was performed, and the amount of BCL-XL bound BIM, BID and BIK was compared to the amount of BCL-XL bound to PUMA. The relative amount of BCL-XL bound to PUMA was set as 1. FIG. 27G) Western blotting for BCL-XL using equal amounts of already IP'd protein lysates from 0Gy and 15Gy irradiated GBM39 cells 24 hours after irradiation. Equal concentrations from proteins BCL-XL, PUMA, BIM, BID, and BIK were IP'd from lysates acquired from GBM39 cells 24 hours after radiation. FIG. 27H) Densitometric analysis of the western blots compares the binding of BH-3 proteins to BCL-XL in GBM39 cell line at 0Gy and 15Gy conditions.

FIGS. 28A-28G: PUMA preferentially interacts with BCL-XL and BCL-W. FIG. 28A) Western blotting for PUMA using equal amounts of already IP'd protein lysates from non-irradiated GBM39 cells. Equal concentrations from proteins PUMA, BCL2, BCL2L1 (AKA: BCL-XL), BCL2L2 (AKA: BCL-W), and MCL1 were immune precipitated from lysates acquired from non-irradiated GBM39 cells. 4× lysate concentration was used in the non-irradiated group to show a good level of PUMA given the relatively low level of PUMA in non-radiated cells that normally needs a damage-inducing agent as IR to be well detected. FIG. 28B) Densitometric analysis of the western blots' bands was performed, and the amount of PUMA bound to BCL-XL, BCL-W, and MCL1 was normalized to the amount of PUMA bound to BCL2. The relative amount of PUMA bound to BCL2 was set as 1. FIG. 28C) Western blotting for PUMA using equal amounts of already IP'd protein lysates from 15Gy irradiated GBM39 cells, 24 hours after irradiation. Equal concentrations from proteins PUMA, BCL2, BCL-XL, BCL-W, and MCL1 were IP'd from lysates acquired from 15Gy irradiated GBM39 cells 24 hours after irradiation. FIG. 28D) Densitometric analysis of the western blots was performed, and the amount of PUMA bound to BCL-XL, BCL-W, and MCL1 was normalized to the amount of PUMA bound to BCL-2. The relative amount of PUMA bound to BCL2 was set as 1. FIG. 28E) Western blot confirms the knockdown of BCL-W in non-irradiated (0Gy) and 15Gy irradiated GBM39. FIG. 28F) Cell titer-glo assay demonstrating the cellular viability of GBM39 (0Gy vs. 15Gy) cells in response to the knock-down of BCL-W. Values are normalized to results acquired from the same GBM39 (0Gy vs. 15Gy) cells treated with a scrambled shRNA. Unpaired t-test was used for statistical analysis, error bars representing SD values, were measured from three different samples (biological triplicate) as shown by the individually plotted data points and P values are as shown in the graph. FIG. 28G) Caspase-3 assay demonstrating the detected enzymatic activity of caspase-3 enzyme of GBM39 (0Gy vs. 15Gy) cells in response to the knock-down of BCL-W. Values are normalized to results acquired from the same GBM39 (0Gy vs. 15Gy) cells treated with a scrambled shRNA. Unpaired t-test was used for statistical analysis, error bars representing SD values, were measured from three different samples (biological triplicate) as shown by the individually plotted data points and P values are as shown in the graph.

FIGS. 29A-29F: PUMA binds to BAX after BCL-XL knock-down leading to apoptotic cell death in GBM cells. FIG. 29A) Western blotting for PUMA using equal amounts of already IP'd protein lysates from 15Gy irradiated GBM39 cells. Equal concentrations from proteins BCL-XL, PUMA, and BAX were IP'd from lysates acquired from 15Gy irradiated GBM39 cells as described in the Materials and Methods. FIG. 29B) Western blotting for PUMA using equal amounts of already IP'd protein lysates from 15Gy radiated GBM39 cells with an already Knocked-down BCL-XL. Equal concentrations from proteins BCL-XL, PUMA, and BAX were IP'd from lysates acquired from 15Gy radiated GBM39 cells with an already knocked-down BCL-XL. FIG. 29C) Densitometric analysis of the western blots was performed as described in the Materials and Methods, and the amount of PUMA bound to BAX was compared to the amount of PUMA bound to BCL-XL. The relative amount of PUMA bound to BCL-XL was set as 1. FIG. 29D) Densitometric analysis of the western blots was performed, and the amount of PUMA bound to BAX was compared to the amount of PUMA bound to BCL-XL. The relative amount of PUMA bound to BCL-XL was set as 1. FIG. 29E) Cell titer-glo assay demonstrating the cellular viability of GBM39 (0Gy vs. 15Gy) cells in response to the knock-down of BCL-XL. Values are normalized to results acquired from the same GBM39 (0Gy vs. 15Gy) cells treated with a scrambled shRNA. Unpaired t-test was used for statistical analysis, error bars representing SD values, were measured from three different samples (biological triplicate) as shown by the individually plotted data points and P values are as shown in the graph. FIG. 29F) Caspase-3 assay demonstrating the caspase-3 enzymatic activity of GBM39 (0Gy vs. 15Gy) cells in response to the knock-down of BCL-XL. Values are normalized to results acquired from the same GBM39 (0Gy vs. 15Gy) cells treated with a scrambled shRNA. Unpaired t-test was used for statistical analysis, error bars representing SD values, were measured from three different samples (biological triplicate) as shown by the individually plotted data points and P values are as shown in the graph.

FIGS. 30A-30H: BAX and PUMA are critical to inducing apoptosis in GBM cells in response to BCL-XL knock-down. FIG. 30A) Western blot confirmation of the knock-out of BCL-XL and BAX in 2 different biological groups (colonies) of GBM39 cells compared to GAPDH as a loading control. FIG. 30B) Western Blotting for PUMA using equal amounts of already IP'd protein lysates from non-irradiated vs. 15Gy-irradiated GBM39 cells. Equal concentrations from proteins BCL-XL, PUMA, and BAX were IP'd from lysates acquired from these GBM39 cells. FIG. 30C) Cell titer-glo assay demonstrating the cellular viability of GBM39 (0Gy vs. 15Gy) cells in response to the knock-out of BCL-XL and BAX. Values are normalized to results acquired from the same GBM39 (0Gy vs. 15Gy) cells treated with a scrambled sgRNA. Unpaired t-test was used for statistical analysis, error bars representing SD values, were measured from three different samples (Biological triplicate) as shown by the individually plotted data points and P values are as shown in the graph. FIG. 30D) Caspase-3 assay demonstrating the caspase-3 enzymatic activity in GBM39 (0Gy vs. 15Gy) cells in response to the knock-out of BCL-XL and BAX. Values are normalized to results acquired from the same GBM39 (0Gy vs. 15Gy) cells treated with a scrambled sgRNA. Unpaired t-test was used for statistical analysis, error bars representing SD values, were measured from three different samples (biological triplicate) as shown by the individually plotted data points and P values are as shown in the graph. FIG. 30E) Western blot confirmation of the knock-out of PUMA in GBM39 cells compared to GAPDH as a loading control. FIG. 30F) Western blot confirmation of the knock-down of BCL-XL in GBM39 cells already knocked-out of PUMA compared to GAPDH as a loading control. FIG. 30G) Cell titer-glo assay demonstrating the cellular viability of GBM39 (0Gy vs. 15Gy) cells in response to the knockdown of BCL-XL in the cells already knocked-out of PUMA. Values are normalized to results acquired from the same GBM39 (0Gy vs. 15Gy) cells treated with a scrambled shRNA. Unpaired t-test was used for statistical analysis, error bars representing SD values, were measured from three different samples (biological triplicate) as shown by the individually plotted data points and P values are as shown in the graph. FIG. 30H) Caspase-3 assay demonstrating the caspase-3 enzymatic activity in GBM39 (0Gy vs. 15Gy) cells in response to the knock-down of BCL-XL. Values are normalized to results acquired from the same GBM39 (0Gy vs. 15Gy) cells treated with a scrambled shRNA. Unpaired t-test was used for statistical analysis, error bars representing SD values, were measured from three different samples (biological triplicate) as shown by the individually plotted data points and P values are as shown in the graph.

FIG. 31: A schematic of an exemplary mechanism for how GBM cells can upregulate PUMA and BCL-XL in response to radiotherapy. PUMA: p53 upregulated modulator of apoptosis; BCL-XL: B-cell lymphoma extra-large protein; BAX: B-cell lymphoma associated X protein.

### DETAILED DESCRIPTION

This document provides methods and materials for treating mammals (e.g., humans) having cancer (e.g., a CNS cancer such as a latent CNS cancer). In some cases, one or more (e.g., one, two, three, four, or more) inhibitors of a Bcl-xL polypeptide can be used to treat mammals (e.g., humans) having cancer (e.g., a CNS cancer such as a latent CNS cancer). For example, one or more inhibitors of a Bcl-xL polypeptide can be administered to a mammal (e.g., a human) having cancer (e.g., a CNS cancer such as a latent CNS cancer) to treat the mammal.

In some cases, one or more (e.g., one, two, three, four, or more) inhibitors of a Bcl-xL polypeptide can be administered to a mammal (e.g., a human) in need thereof (e.g., a human having cancer (e.g., a CNS cancer such as a latent CNS cancer) to delay or prevent the development of a recurrent CNS cancer (e.g., a glioma such as a GBM). In some cases, the methods and materials described herein can be used to delay the development of a recurrent CNS cancer (e.g., a glioma such as a GBM) by, for example, 10, 20, 30, 40, 50, 60, 70, 80, 90, 95, or more percent. In some cases, the methods and materials described herein can be used to delay the development of a recurrent CNS cancer (e.g., a glioma such as a GBM) by at least 1 month. In some cases, the methods and materials described herein can be used to delay the development of a recurrent CNS cancer (e.g., a glioma such as a GBM) by from about 1 month to about 10 years (e.g., from about 1 month to about 10 years, from about 1 month to about 9 years, from about 1 month to about 8 years, from about 1 month to about 7 years, from about 1 month to about 6 years, from about 1 month to about 5 years, from about 1 month to about 4 years, from about 1 month to about 3 years, from about 1 month to about 2 years, from about 1 month to about 1 year, from about 1 month to about 6 months, from about 6 months to about 10 years, from about 1 year to about 10 years, from about 2 years to about 10 years, from about 3 years to about 10 years, from about 4 years to about 10 years, from about 5 years to about 10 years, from about 6 years to about 10 years, from about 7 years to about 10 years, from about 8 years to about 10 years, from about 9 years to about 10 years, from about 6 months to about 9 years, from about 1 year to about 8 years, from about 2 years to about 7 years, from about 3 years to about 6 years, from about 4 years to about 5 years, from about 1 month to about 2 years, from about 2 years to about 4 years, from about 4 years to about 6 years, or from about 6 years to about 8 years).

In some cases, one or more (e.g., one, two, three, four, or more) inhibitors of a Bcl-xL polypeptide can be administered to a mammal (e.g., a human) in need thereof (e.g., a human having cancer (e.g., a CNS cancer such as a latent CNS cancer) to reduce or eliminate the number of cancer cells present within a mammal. For example, the methods and materials described herein can be used to reduce the number of cancer cells present within a mammal having cancer (e.g., a CNS cancer such as a latent CNS cancer) by, for example, 10, 20, 30, 40, 50, 60, 70, 80, 90, 95, or more percent. For example, the methods and materials described herein can be used to reduce the size (e.g., volume) of one or more tumors present within a mammal having cancer (e.g., a CNS cancer such as a latent CNS cancer) by, for example, 10, 20, 30, 40, 50, 60, 70, 80, 90, 95, or more percent.

In some cases, one or more (e.g., one, two, three, four, or more) inhibitors of a Bcl-xL polypeptide can be administered to a mammal (e.g., a human) in need thereof (e.g., a human having cancer (e.g., a CNS cancer such as a latent CNS cancer) to improve survival of the mammal. For example, disease-free survival (e.g., recurrence-free survival) can be improved using the methods and materials described herein. For example, recurrence-free survival can be improved using the methods and materials described herein. In some cases, the methods and materials described herein can be used to improve the survival of a mammal having cancer (e.g., a CNS cancer such as a latent CNS cancer) by, for example, 10, 20, 30, 40, 50, 60, 70, 80, 90, 95, or more percent.

In some cases, one or more (e.g., one, two, three, four, or more) inhibitors of a Bcl-xL polypeptide can be administered to a mammal (e.g., a human) in need thereof (e.g., a human having cancer (e.g., a CNS cancer such as a latent CNS cancer) to delay or prevent the onset of one or more symptoms of a CNS cancer (e.g., a glioma such as a GBM) and/or one or more complications associated with a CNS cancer (e.g., a glioma such as a GBM). Examples of symptoms of a CNS cancer (e.g., a glioma such as a GBM) and complications associated with a CNS cancer (e.g., a glioma such as a GBM) include, without limitation, headaches, vomiting, seizures, cranial nerve disorders (e.g., as a result of increased intracranial pressure), loss of vision, pain, weakness, numbness (e.g., numbness in the extremities), language impairment, and impairment of consciousness. In some cases, the methods and materials described herein can be used to delay the onset of one or more symptoms of a CNS cancer (e.g., a glioma such as a GBM) and/or one or more complications associated with a CNS cancer (e.g., a glioma such as a GBM) by, for example, 10, 20, 30, 40, 50, 60, 70, 80, 90, 95, or more percent.

Any appropriate mammal having cancer (e.g., a CNS cancer such as a latent CNS cancer) can be treated as described herein (e.g., by administering one or more inhibitors of a Bcl-xL polypeptide). Examples of mammals having cancer (e.g., a CNS cancer such as a latent CNS cancer) that can be treated as described herein include, without limitation, humans, non-human primates (e.g., monkeys), dogs, cats, horses, cows, pigs, sheep, mice, and rats. In some cases, a mammal having cancer (e.g., a CNS cancer such as a latent CNS cancer) can be a mammal (e.g., a human) that received one or more cancer treatments (e.g., radiation therapies and chemotherapies) for a prior cancer (e.g., a prior CNS cancer). For example, a mammal having cancer can have been previously treated and can have responded to the previous treatment such that the previous treatment was effective to reduce the cancer such that the mammal is in remission or partial remission.

When treating a mammal (e.g., a human) having cancer (e.g., a CNS cancer such as a latent CNS cancer) as described herein (e.g., by administering one or more inhibitors of a Bcl-xL polypeptide), the cancer can be any type of cancer. In some cases, a cancer can be a chemotherapy resistant (e.g., temozolomide (TZM)-resistant) cancer. In some cases, a cancer can be a radiation resistant cancer. In some cases, a cancer can include one or more solid tumors. In some cases, a cancer can be a blood cancer. In some cases, a cancer can be a primary cancer. In some cases, a cancer can be a malignant pre-metastatic lesion. In some cases, a cancer can be a metastatic cancer. In some cases, a cancer can include one or more senescent cancer cells. For example, a cancer can include one or more cells in which senescence is induced by one or more inhibitors of a Bcl-xL polypeptide. A cancer can be any grade cancer (e.g., Grade I, Grade II, Grade III, and Grade IV). Examples of cancers that can be treated as described herein include, without limitation, CNS cancers (e.g., gliomas (e.g., brain stem gliomas, GBMs, astrocytomas, oligodendrogliomas, oligoastrocytomas, and ependymomas), medulloblastomas, and meningiomas), lymphomas, breast cancers, lung cancers, colon cancers, ovarian cancers, kidney cancers, and lymph node cancers. When a cancer is a CNS cancer, the CNS cancer can include one or more astrocytic tumors, one or more oligodendroglial tumors, one or more ependymal tumors, one or more CNS lymphomas, one or more pineal parenchymal tumors, and/or one or more meningeal tumors.

In some cases, the methods described herein can include identifying a mammal (e.g., a human) as having cancer (e.g., a CNS cancer such as a latent CNS cancer). Any appropriate method can be used to identify a mammal as having cancer (e.g., a CNS cancer such as a latent CNS cancer). For example, medical history (e.g., a history of having had a prior cancer such as a prior CNS cancer), neurological examinations (e.g., to check vision, hearing, balance, coordination, strength, and/or reflexes), imaging techniques such as magnetic resonance imaging (MRI), magnetic resonance spectroscopy, computed tomography (CT) scanning, and positron emission tomography (PET) scanning (e.g., to determine the location and size of a brain tumor), and/or biopsy techniques (e.g., liquid biopsy techniques to detect the presence of a metabolomic and/or a genomic signature of glioma) can be used to identify mammals (e.g., humans) having cancer (e.g., a CNS cancer such as a latent CNS cancer).

A mammal (e.g., a human) having cancer (e.g., a CNS cancer such as a latent CNS cancer) can be administered or instructed to self-administer one or more (e.g., one, two, three, four, or more) inhibitors of a Bcl-xL polypeptide. An inhibitor of a Bcl-xL polypeptide can be an inhibitor of Bcl-xL polypeptide activity or an inhibitor of Bcl-xL polypeptide expression. Examples of compounds that can inhibit Bcl-xL polypeptide activity include, without limitation, anti-Bcl-xL antibodies (e.g., neutralizing anti-Bcl-xL antibodies and anti-Bcl-xL-antibody-drug conjugates), small molecules that target (e.g., target and bind) to a Bcl-xL polypeptide, and agents that can reduce or eliminate binding of pro-apoptotic proteins (e.g., PUMA, BID, or BIM) to Bcl-xL. In some cases, an inhibitor of Bcl-xL polypeptide activity that can be used as described herein also can have senolytic activity. Examples of inhibitors of Bcl-xL polypeptide activity that can be used as described herein include, without limitation, navitoclax, A1331852, A1155463, and WEHI-539. In some cases, an inhibitor of Bcl-xL polypeptide activity that can be used as described herein does not inhibit BCL-2.

Examples of compounds that can reduce or eliminate Bcl-xL polypeptide expression include, without limitation, nucleic acid molecules designed to induce RNA interference of Bcl-xL polypeptide expression (e.g., a siRNA molecule or a shRNA molecule), antisense molecules, miRNAs, and nucleic acid molecules designed to induce CRISPR interference (CRISPRi) of Bcl-xL polypeptide expression (e.g., a guide RNA (gRNA) molecule complexed with a Cas9 polypeptide such as catalytically dead Cas9 polypeptide). Examples of nucleic acid molecules designed to induce CRISPRi of a Bcl-xL polypeptide can be as shown in the table below.

TABLE A
Inhibitors of Bcl-xL polypeptides

| Inhibitor | SEQ ID NO |
|---|---|
| AGGCGACGAGTTTGAACTG | 1 |
| GTGGAAGAGAACAGGACTG | 2 |
| CTCTGATATGCTGTCCCTG | 3 |

In some cases, nucleic acid molecules designed to induce RNAi against Bcl-xL polypeptide expression can be as described elsewhere (see, e.g., ON-TARGETplus™ Human BCL2L1 siRNA; Jackson et al., RNA, 12(7):1197-1205 (2006); Birmingham et al., Nature Methods, 3(3):199-204 (2006); and Anderson et al., RNA, 14(5):853-861 (2008)).

Additional nucleic acid molecules designed to induce RNAi against Bcl-xL polypeptide expression can be designed based on any appropriate nucleic acid encoding a Bcl-xL polypeptide sequence. Examples of nucleic acids encoding a Bcl-xL polypeptide sequence include, without limitation, those set forth in National Center for Biotechnology Information (NCBI) accession no. AA488236.1, accession no. AI872556.1, accession no. AI872557.1, accession no. AK290968.1, or accession no. AY263335.1.

In cases where a compound that can reduce or eliminate Bcl-xL polypeptide expression is a nucleic acid molecule designed to induce RNA interference of Bcl-xL polypeptide expression, the nucleic acid molecule can be in the form of a nucleic acid vector (e.g., a viral vector or a non-viral vector).

When a vector used to deliver a nucleic acid molecule designed to induce RNA interference of Bcl-xL polypeptide expression to a mammal (e.g., a human) having cancer (e.g., a CNS cancer such as a latent CNS cancer) is a viral vector, any appropriate viral vector can be used. Examples of virus-based vectors that can be used to deliver a nucleic acid molecule designed to induce RNA interference of Bcl-xL polypeptide expression described herein to a mammal include, without limitation, virus-based vectors based on a lentivirus, virus-based vectors based on an adenovirus, and virus-based vectors based on an adeno-associated virus.

When a vector used to deliver a nucleic acid molecule designed to induce RNA interference of Bcl-xL polypeptide expression to a mammal (e.g., a human) having cancer (e.g., a CNS cancer such as a latent CNS cancer) is a non-viral vector, any appropriate non-viral vector can be used. In some cases, a non-viral vector can be an expression plasmid (e.g., a cDNA expression vector).

In some cases, an inhibitor of a Bcl-xL polypeptide can be as described elsewhere (see, e.g., Wang et al., ACS Med Chem. Lett., 11(10):1829-1836 (2020); Tse et al., Cancer Res., 68(9):3421-8 (2008); and Lessene et al., Nat. Chem. Biol., 9(6):390-7 (2013)).

In some cases, an inhibitor of a Bcl-xL polypeptide, when administered to a mammal (e.g., a human) having cancer (e.g., a CNS cancer such as a latent CNS cancer), can cross the blood brain barrier of the mammal.

In cases where a mammal (e.g., a human) having cancer (e.g., a CNS cancer such as a latent CNS cancer) has received one or more cancer treatments (e.g., radiation therapies and chemotherapies) for a prior cancer (e.g., a prior CNS cancer), one or more inhibitors of a Bcl-xL polypeptide can be administered to the mammal at any time following the prior cancer treatment(s) for the prior cancer. For example, a prior CNS cancer can have been treated from about 0 days to about 120 months (e.g., from about 0 days to about 60 months, from about 0 days to about 36 months, from about 0 days to about 24 months, from about 0 days to about 12 months, from about 0 days to about 9 months, from about 0 days to about 6 months, from about 0 days to about 3 months, from about 7 days to about 120 months, from about 3 months to about 120 months, from about 6 months to about 120 months, from about 12 months to about 120 months, from about 24 months to about 120 months, from about 36 months to about 120 months, from about 60 months to about 120 months, from about 7 days to about 60 months, from about 3 months to about 24 months, from about 6 months to about 12 months, from about 7 days to about 1 month, from about 1 month to about 3 months, from about 3 months to about 6 months, from about 6 months to about 12 months, from about 12 months to about 24 months, or from about 24 months to about 60 months) prior to administering one or more inhibitors of a Bcl-xL polypeptide described herein. In cases where a mammal (e.g., a human) having received radiation therapy for a prior cancer (e.g., a prior CNS cancer) is administered one or more inhibitors of a Bcl-xL polypeptide, the one or more inhibitors of a Bcl-xL polypeptide can be administered from about 0 days to about 12 months (e.g., from about 0 days to about 9 months, from about 0 days to about 6 months, from about 0 days to about 4 months, from about 0 days to about 3 months, from about 7 days to about 12 months, from about 2 months to about 12 months, from about 4 months to about 12 months, from about 6 months to about 12 months, from about 9 months to about 12 months, from about 7 days to about 9 months, from about 1 month to about 6 months, or from about 3 months to about 4 months) post-radiation therapy.

In some cases, a mammal (e.g., a human) having cancer (e.g., a CNS cancer such as a latent CNS cancer) that has received one or more cancer treatments (e.g., radiation therapies and chemotherapies) for a prior cancer (e.g., a prior CNS cancer) can be identified as having responded to the previous treatment (e.g., can be identified as being in remission or partial remission) prior to being treated as described herein (e.g., by administered one or more inhibitors of a Bcl-xL polypeptide). For example, a mammal having cancer can have been previously treated for cancer (e.g., can have previously received one or more radiation therapies and/or one or more chemotherapies), can have been identified as having responded to the previous treatment (e.g., can be identified as being in remission or partial remission), and can be administered one or more inhibitors of a Bcl-xL polypeptide at least 1 month after the prior cancer treatment(s) for the prior cancer. In some cases, a mammal having cancer can have been previously treated for cancer (e.g., can have previously received one or more radiation therapies and/or one or more chemotherapies), can have been identified as having responded to the previous treatment (e.g., can be identified as being in remission or partial remission), and can be administered one or more inhibitors of a Bcl-xL polypeptide from about 1 month to about 10 years (e.g., about 1 month to about 10 years, about 1 month to about 8 years, about 1 month to about 6 years, about 1 month to about 5 years, about 1 month to about 4 years, about 1 month to about 3 years, about 1 month to about 2 years, about 1 month to about 18 months, about 1 month to about 12 months, about 1 month to about 6 months, about 6 months to about 10 years, about 12 months to about 10 years, about 18 months to about 10 years, about 2 years to about 10 years, about 3 years to about 10 years, about 4 years to about 10 years, about 5 years to about 10 years, about 7 years to about 10 years, about 6 months to about 5 years, about 12 months to about 18 months, about 1 year to about 3 years, about 3 years to about 5 years, about 5 years to about 7 years, or about 7 years to about 9s years) after having received the prior cancer treatment(s).

In some cases when a mammal (e.g., a human) having cancer (e.g., a CNS cancer such as a latent CNS cancer) has received one or more cancer treatments (e.g., radiation therapies and chemotherapies) for a prior cancer (e.g., a prior CNS cancer) and is identified as being in remission or partial remission prior to being treated as described herein (e.g., by administered one or more inhibitors of a Bcl-xL polypeptide), the mammal does not receive any other cancer treatments between the previous treatment and being administered one or more inhibitors of a Bcl-xL polypeptide described herein. For example, a mammal having been previously treated for cancer (e.g., having previously received one or more radiation therapies and/or one or more chemotherapies) can be administered one or more inhibitors of a Bcl-xL polypeptide after having not been treated for cancer for at least 1 month. In some cases, a mammal having been previously treated for cancer (e.g., having previously received one or more radiation therapies and/or one or more chemotherapies) can be administered one or more inhibitors of a Bcl-xL polypeptide after having not been treated for cancer for from about 1 month to about 10 years (e.g., about 1 month to about 10 years, about 1 month to about 8 years, about 1 month to about 6 years, about 1 month to about 5 years, about 1 month to about 4 years, about 1 month to about 3 years, about 1 month to about 2 years, about 1 month to about 18 months, about 1 month to about 12 months, about 1 month to about 6 months, about 6 months to about 10 years, about 12 months to about 10 years, about 18 months to about 10 years, about 2 years to about 10 years, about 3 years to about 10 years, about 4 years to about 10 years, about 5 years to about 10 years, about 7 years to about 10 years, about 6 months to about 5 years, about 12 months to about 18 months, about 1 year to about 3 years, about 3 years to about 5 years, about 5 years to about 7 years, or about 7 years to about 9s years).

In some cases, one or more inhibitors of a Bcl-xL polypeptide can be formulated into a composition (e.g., a pharmaceutically acceptable composition) for administration to a mammal (e.g., a human) having cancer (e.g., a CNS cancer such as a latent CNS cancer). For example, one or more inhibitors of a Bcl-xL polypeptide can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, when a composition containing one or more inhibitors of a Bcl-xL polypeptide is administered to a mammal (e.g., a human) having cancer (e.g., a CNS cancer such as a latent CNS cancer), the composition can be designed for oral or parenteral (including, without limitation, a subcutaneous, intramuscular, intravenous, intradermal, intra-cerebral, intrathecal, or intraperitoneal (i.p.) injection) administration to the mammal. Compositions suitable for oral administration include, without limitation, liquids, tablets, capsules, pills, powders, gels, and granules. In some cases, compositions suitable for oral administration can be in the form of a food supplement. In some cases, compositions suitable for oral administration can be in the form of a drink supplement. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition containing one or more inhibitors of a Bcl-xL polypeptide can be administered to a mammal (e.g., a human) having cancer (e.g., a CNS cancer such as a latent CNS cancer) in any appropriate amount (e.g., any appropriate dose). An effective amount of a composition containing one or more inhibitors of a Bcl-xL polypeptide can be any amount that can treat a mammal having cancer (e.g., a CNS cancer such as a latent CNS cancer) as described herein without producing significant toxicity to the mammal. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the CNS cancer in the mammal being treated may require an increase or decrease in the actual effective amount administered.

A composition containing one or more inhibitors of a Bcl-xL polypeptide can be administered to a mammal (e.g., a human) having cancer (e.g., a CNS cancer such as a latent CNS cancer) in any appropriate frequency. The frequency of administration can be any frequency that can treat a mammal having cancer (e.g., a CNS cancer such as a latent CNS cancer) without producing significant toxicity to the mammal. For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more inhibitors of a Bcl-xL polypeptide can be administered to a mammal (e.g., a human) having cancer (e.g., a CNS cancer such as a latent CNS cancer) for any appropriate duration. An effective duration for administering or using a composition containing one or more inhibitors of a Bcl-xL polypeptide can be any duration that can treat a mammal having cancer (e.g., a CNS cancer such as a latent CNS cancer) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having cancer (e.g., a CNS cancer such as a latent CNS cancer) can include administering to the mammal one or more inhibitors of a Bcl-xL polypeptide as the sole active ingredient to treat the cancer (e.g., a CNS cancer such as a latent CNS cancer) in the mammal. For example, a composition containing one or more inhibitors of a Bcl-xL polypeptide can include the one or more inhibitors of a Bcl-xL polypeptide as the sole active ingredient in the composition that is effective to treat a mammal having cancer (e.g., a CNS cancer such as a latent CNS cancer).

In some cases, methods for treating a mammal (e.g., a human) having cancer (e.g., a CNS cancer such as a latent CNS cancer) can include administering to the mammal one or more inhibitors of a Bcl-xL polypeptide in the absence of any BCL-2 inhibitor(s) to treat the cancer (e.g., a CNS cancer such as a latent CNS cancer) in the mammal.

In some cases, methods for treating a mammal (e.g., a human) having cancer (e.g., a CNS cancer such as a latent CNS cancer) can include administering to the mammal one or more inhibitors of a Bcl-xL polypeptide in the absence of any Syk inhibitor(s) to treat the cancer (e.g., a CNS cancer such as a latent CNS cancer) in the mammal.

In some cases, methods for treating a mammal (e.g., a human) having cancer (e.g., a CNS cancer such as a latent CNS cancer) can include administering to the mammal one or more inhibitors of a Bcl-xL polypeptide (e.g., an inhibitor of a Bcl-xL polypeptide that also has senolytic activity or an inhibitor of a Bcl-xL polypeptide that lacks senolytic activity) together with one or more (e.g., one, two, three, four, five or more) senotherapeutic agents. In some cases, a senotherapeutic agent that can be used in combination with an inhibitor of a Bcl-xL polypeptide as described herein can be a senolytic agent (i.e., an agent having the ability to induce cell death in senescent cells). In some cases, a senotherapeutic agent that can be used in combination with an inhibitor of a Bcl-xL polypeptide as described herein can be a senomorphic agent (i.e., an agent having the ability to suppress senescent phenotypes without cell killing). In some cases, a senotherapeutic agent that can be used in combination with an inhibitor of a Bcl-xL polypeptide as described herein can also have the ability to inhibit a Bcl-xL polypeptide. In some cases, a senotherapeutic agent that can be used in combination with an inhibitor of a Bcl-xL polypeptide as described herein can lack the ability to inhibit a Bcl-xL polypeptide.

Examples of senotherapeutic agents that can be administered together with one or more inhibitors of a Bcl-xL polypeptide described herein and that have the ability to inhibit a Bcl-xL polypeptide include, without limitation, navitoclax, A1331852, A1155463, and WEHI-539. Examples of senotherapeutic agents that can be administered together with one or more inhibitors of a Bcl-xL polypeptide described herein and that lack the ability to inhibit a Bcl-xL polypeptide include, without limitation, saracatinib, onalespib, AMB232, piperlongumine, fisetin, quercetin, dasatinib, and any combinations thereof. In cases where one or more inhibitors of a Bcl-xL polypeptide described herein are used in combination with one or more senotherapeutic agents, the one or more senotherapeutic agents can be administered at the same time (e.g., in a single composition containing both one or more inhibitors of a Bcl-xL polypeptide described herein and the one or more senotherapeutic agents) or independently. For example, one or more inhibitors of a Bcl-xL polypeptide described herein can be administered first, and the one or more senotherapeutic agents administered second, or vice versa. In addition, in cases where an inhibitor of a Bcl-xL polypeptide is used in combination with a senotherapeutic agent, the inhibitor of a Bcl-xL polypeptide can be different from the senotherapeutic agent even though that inhibitor of a Bcl-xL polypeptide may also have senolytic activity. For example, navitoclax can be administered first as an inhibitor of a Bcl-xL polypeptide that also has senolytic activity, and A1331852 can be administered second as a senotherapeutic agent that also has the ability to inhibit a Bcl-xL polypeptide.

In some cases, methods for treating a mammal (e.g., a human) having cancer (e.g., a CNS cancer such as a latent CNS cancer) can include administering to the mammal one or more inhibitors of a Bcl-xL polypeptide together with one or more (e.g., one, two, three, four, five or more) additional anti-cancer agents (e.g., chemotherapeutic agents) used to treat a CNS cancer. In some cases, administering one or more inhibitors of a Bcl-xL polypeptide to a mammal (e.g., a human) having cancer (e.g., a CNS cancer such as a latent CNS cancer) can sensitize cancer cells in the CNS cancer to one or more anti-cancer agents (e.g., chemotherapeutic agents such as temozolomide). In some cases, an anti-cancer agent can be an alkylating agent. In some cases, an anti-cancer agent can be an immunotherapeutic agent. In some cases, an anti-cancer agent can be a NAMPT inhibitor. In some cases, an anti-cancer agent can be a XPO1 inhibitor. In some cases, an anti-cancer agent can be a MDM2 inhibitor. Examples of anti-cancer agents that can be administered together with one or more inhibitors of a Bcl-xL polypeptide described herein include, without limitation, temozolomide (e.g., TEMODAR®), selinexor (e.g., XPOVIO®), APO866, AMG-232, RG7388, GMX1778, and any combinations thereof. In cases where one or more inhibitors of a Bcl-xL polypeptide described herein are used in combination with additional agents used to treat a CNS cancer, the one or more additional agents can be administered at the same time (e.g., in a single composition containing both one or more inhibitors of a Bcl-xL polypeptide described herein and the one or more additional agents) or independently. For example, one or more inhibitors of a Bcl-xL polypeptide described herein can be administered first, and the one or more additional agents administered second, or vice versa.

In some cases, methods for treating a mammal (e.g., a human) having cancer (e.g., a CNS cancer such as a latent CNS cancer) can include administering to the mammal one or more inhibitors of a Bcl-xL polypeptide together with one or more (e.g., one, two, three, four, five or more) additional therapies used to treat a CNS cancer. Examples of therapies that can be used to treat a CNS cancer include, without limitation, surgery, radiation therapy, laser interstitial thermal therapy, and focused ultrasound. In cases where one or more inhibitors of a Bcl-xL polypeptide described herein are used in combination with one or more additional therapies used to treat a CNS cancer, the one or more additional therapies can be performed at the same time or independently of the administration of the one or more inhibitors of a Bcl-xL polypeptide described herein. For example, the one or more inhibitors of a Bcl-xL polypeptide described herein can be administered before, during, or after the one or more additional therapies are performed.

The invention will be further described in the following examples, which do not limit the scope of the invention described in the claims.

### EXAMPLES

### Example 1: Selective Vulnerability of Senescent Glioblastoma Cells to Bcl-XL Inhibition

Radiation and TMZ endow glioma with increased dependence upon Bcl-XL, blockade of which may facilitate ablation of latent glioma cells that survived prior cytotoxic therapy.

### Materials and Methods

### Cell Culture

Human patient-derived glioblastoma lines were utilized and were cultured according to established protocols. Tumor lines maintained as patient-derived xenografts (PDX) are from the National PDX resource. Such lines are designated as “GBM-6, 10, 12, 39, 76, 123, 164, or 196”. Implantation of patient-derived glioblastoma cells, serial passage of flank tumor xenografts, and short-term explant culturing were done as described elsewhere, with some lines maintained in serum-containing media, and some lines maintained in serum free media as noted in Table 2 (see, for example, Carlson et al., Current protocols in pharmacology. Chapter 14(14):Unit 14.16 (2011)). Some cell lines were maintained from time of harvest as in vitro cell lines rather than PDX lines, as described elsewhere (see, for example, Himes et al., Neuro-oncology. 22(7):967-978 (2020)). Such lines are designated as dBT114, dBT116, dBT120, dBT132 (dBT=differentiated brain tumor). Culture conditions were unchanged after induction of senescence.

### Senescence Induction

After plating, cells were maintained in 10 cm culture dishes for 2-4 days until >50% confluent. Cells were then treated with TMZ for 7 days or radiation (cesium gamma radiator) with varying doses. Most experiments were performed after use of 15 Gy to induce senescence. Radiation and TMZ each caused death of a variable percentage of cells in the ensuing days. “Senescent” tumor cells used for experiments are designated by those that survive following TMZ or radiation treatments. Except for radiation doses of 4 Gy or below, no visible proliferation occurred within 1 month after treatment with TMZ or radiation. For initial screening of senolytic drugs, cells were maintained for at least 20 days after radiation or TMZ prior to re-plating cells into black walled optical 96-well plates (5000 cells/well) for doing drug treatment.

### Analysis of Cell Proliferation

0, 4, 8, 15, and 20 Gy radiation was delivered as described above. Three days thereafter, cells then plated in 96-well plates. After allowing cells to adhere overnight, they were then placed into the IncuCyte (Thermo Scientific Series 8000 WJ Incubator) and images were captured every four hours for automated quantification of cellular confluence.

### Senolytic Drug Screen

Human glioblastoma cells were exposed to 10, 15 or 20 Gy radiation as indicated above. All GBM lines tested (Table 2) yielded a subpopulation of surviving non-proliferative cells used for subsequent experiments, with the exception of GM43 which yielded insufficient surviving cells for senescence experiments. All senolytic drugs used in this study were dissolved in DMSO (Table 1). Control (0 nM) cells were treated with the same dose of DMSO as cells with the highest drug concentration. Unless specified otherwise, cells were maintained in drug-containing media for four days prior to evaluation of cell viability using ATP lite, CellTiter-Glo assay according to company protocol (Cat #G7570). For all experiments, luminescence values are normalized to the 0 nM control for that cell line and radiation dose. All dose response graphs are depicted standard error of the mean (SEM) of 3 technical replicates at each concentration. Representative data are shown for experiments performed in independent replicates.

### Evaluation of Radiation Dose, Drug Exposure Time Dependency

To evaluate the radiation dose effect on Bcl-XL inhibitor sensitivity, GBM39 cells were radiated with 0, 1, 2, 4, 8, or 15 Gy. F our days after radiation, cells were plated in black wall 96-well plates. Overnight and drugs added the next day. Seven days after drug treatment, cell viability was measured by CellTiter-Glo. To determine the impact of varying duration of drug exposure, 15 Gy radiated cells were plated 7 days after radiation, where the minimum drug exposure time was 1 hour and the maximum 96 hours. Cells were plated in black-wall 96-well plates, and drugs added the next day as described above. At the designated time-point, drug-containing media was removed and replaced with drug-free media for the remainder of the experiment (one wash with media, during a replacement). The cell viability assay was performed at the end of 96 hours.

qRT-PCR

RNA was extracted from cells previously radiated at 15 Gy at 1, 3 and 7 days. Briefly, cells were washed with phosphate buffered saline (PBS) before being homogenized with TriZol reagent (Invitrogen). RNA precipitation was performed at −20° C. overnight. Resulting RNA pellets were dissolved in RNase-free water and concentration was measured by absorbance at 260 nm (A260) using Nanodrop2000. cDNA synthesis was performed with 1 μg of total RNA using M-MLV reverse transcriptase kit (ThermoFisher) as per the manufacturer's protocol. 25 ng of cDNA was used for real-time PCR with Taqman gene expression assay targeting IL-6 (Hs00174131_m1), Bcl-2 (Hs00608023_m1), Bcl-XL (Hs001691412_m1) on ABI 7500/7500-Fast Real-Time PCR System (Applied Bioscience). The relative expression of each gene was determined by the ΔΔCT method.

### SA-β-Gal Staining

Senescence-associated β-galactosidase staining Kit (Cell Signaling Technology #9860) was used as an indicator of relative senescence after radiation as per the manufacturer's directions. Briefly, cells were fixed for 10 minutes in β-galactosidase fixative Solution (10% 100× Fixative Solution; 90% H2O), and washed with PBS. The cells were then stained with β-Galactosidase Staining Solution (93% lx Staining Solution; 1% 100× Solution A, 1% 100× Solution B, 5% X-gal). Wells without samples were filled with PBS, and the plate was wrapped in parafilm to prevent evaporation. The plate was left overnight in a dry incubator at 37° C. The next day, cells were examined under a microscope for β-gal-positive cells (blue staining). PBS was added to the wells, and the plate was placed on the rocker (speed=30/min) for 5 minutes. The plate was washed three times. Staining was performed in sham and vehicle-treated control, 10 Gy, 15 Gy radiated, and TMZ treated GBM39 cells. To see the senescence maturation over time, beta-Gal staining performed at day 0, 7, and 14 following 15 Gy radiation. For each time points and conditions staining done at multiple wells.

### Protein Analysis by Western Blotting

Cells grown in six-well plates, 10 cm dishes or T-25 flasks were washed with PBS, trypsinized, and collected in 1.5 micro-centrifuge tubes as a cell pellet. The cell pellet was then lysed using lysis buffer composed of 10% RIPA lysis buffer, 4% Protease-Inhibitor cocktail, 1% Phosphatase-Inhibitor cocktail-2, 1% Phosphatase-Inhibitor cocktail-3, and 84% molecular grade water. The cell palate with the lysis buffer then sonicated for 30 minutes in a water bath sonicator (one minute sonication every other minute for a total of 30 minutes). The whole lysate was centrifuged for 10 minutes at the speed of 17,000 g to collect the supernatant as the final protein lysate. The concentration of the final protein lysate was then measured using the BCA kit (ThermoFischer). Proteins extracted from cells using lysis buffer, were separated in an SDS-polyacrylamide gel electrophoresis along with the protein ladder (Life Technology) using 4-12% Bis-Tris gel (ThermoFischer). Proteins were then transferred to a polyvinylidene difluoride (PVDF) membrane (Bio-Rad). The membrane was blocked in 5% fat-free milk (Cell-signaling technology) for 30 minutes, washed three times (5 minutes each) using Tris-buffered saline with tween20 (TBST) and probed with different antibodies (Cell-signaling technology).

Gene Knock-Down Using siRNA

siRNA sequences were either designed manually using commercially available soft wares or purchased as an already designed sequences (Horizon-ThermoFisher). The siRNA was re-suspended in 1×siRNA buffer (Dharmacon) or in any other Nuclease-free solutions. Cells were plated overnight with the optimal density in six-well plates (4×10⁵ per well) or 96-well plates (2×10⁴ per well) in antibiotic-free medium. The next day, the transfection complex was prepared by mixing the siRNA (either for the gene of interest or the negative siRNA control) and Lipofectamine RNAiMAX (Invitrogen) in serum-free medium, 250 μL from the transfection complex were added to each well of the six-well plate and 10 μL for each well in 96-well plate. One-day post transfection RNA was collected to be analyzed by qRT-PCR to confirm gene silencing. 2-3 days post transfection, protein was collected to be analyzed by the SDS-page and western blotting to confirm gene silencing. Three days post transfection, the cell viability in response gene silencing was measured using CellTiter-Glo assay (Promega), and the cell survival ratio was calculated compared to the negatively silenced control cells.

### Statistical Analysis

Multiple linear regressions were used to assess the relationship of drug dose and treatment (e.g. control, radiation, TMZ) with cell inhibition within each cell line. For these models, cell inhibition was the outcome and drug dose, treatment, and the drug dose by treatment interaction were the independent predictors. If the drug dose by treatment interaction was significant (p<0.05), pairwise comparisons were made at each drug dose between treatment groups using a two-sample t-test. Statistical analysis was performed in Microsoft Excel. IC₅₀ was calculated by nonlinear regression (Curve fit) of dose-response inhibition curve by using GraphPad prism 8.4.2.

### Results

### Chemoradiation Induces a Senescence-Like State of Sustained Proliferative Arrest

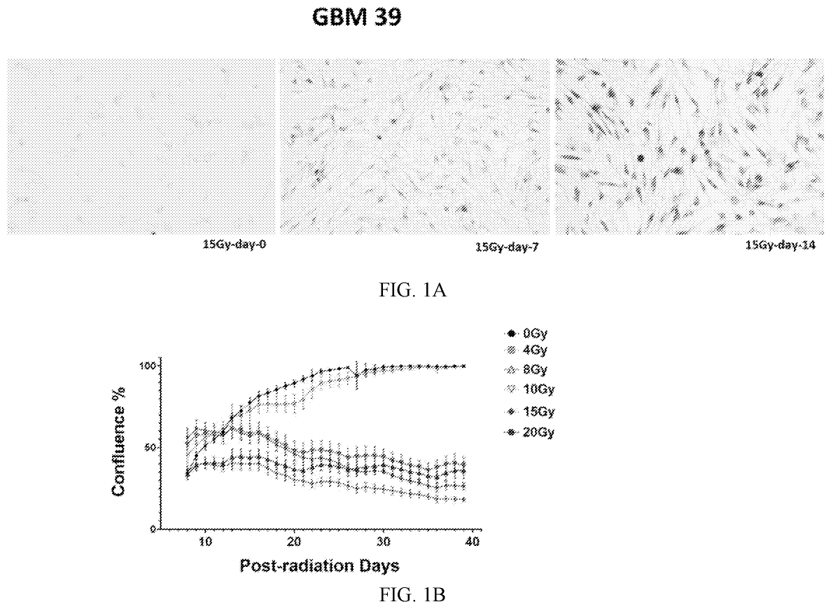

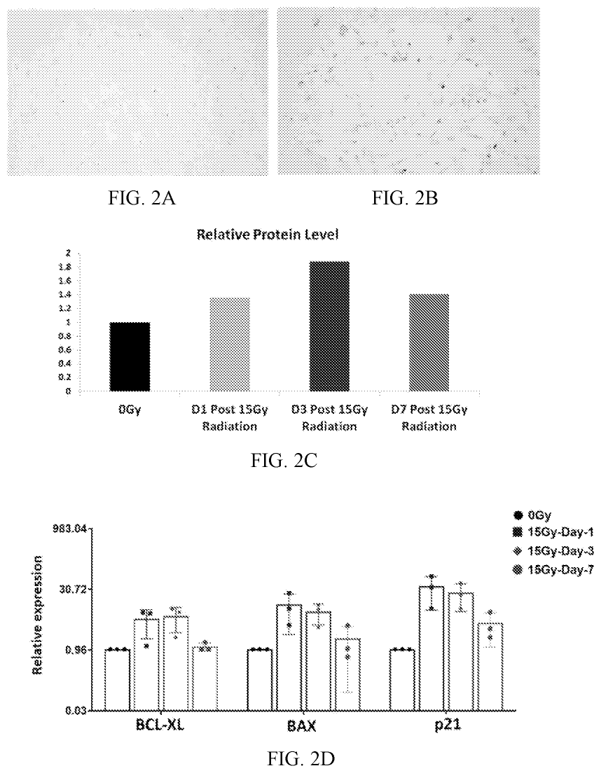

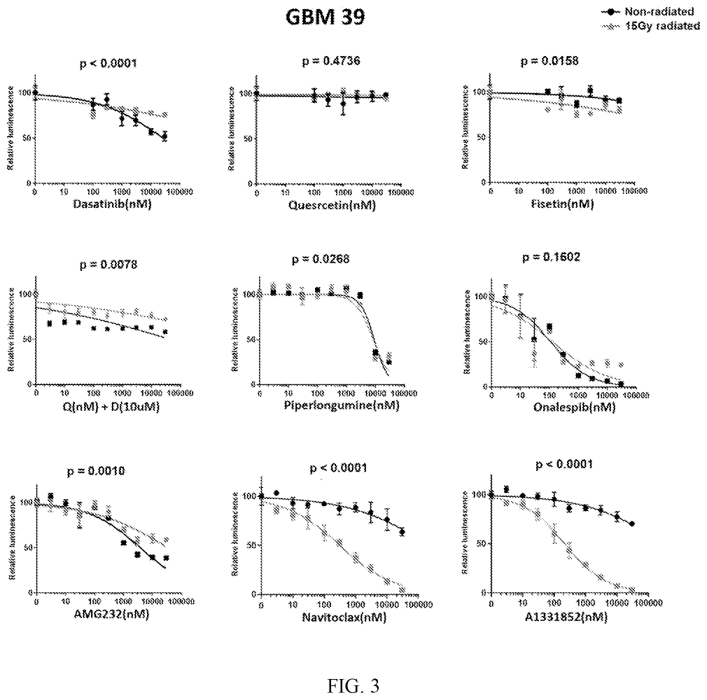

It was hypothesized that glioblastoma could be relatively more sensitive to senolytic ablative therapies when induced into a senescent-like state of proliferative arrest following radiation. Single fraction radiation doses were evaluated in human glioblastoma cell lines and sustained loss of culture expansion with 8 Gy or higher radiation in the GBM39 cell line was observed (FIG. 1A-B). Senescent-like cultures demonstrated evidence of increased SA-β gal staining consistent with a senescent phenotype induced by radiation (FIG. 1A-B, FIG. 2A-C). Higher expression of the senescence-associated gene, p21, and the anti-apoptotic pathway up-regulation, was also apparent in residual cells over seven days following radiation (FIG. 2D).

### Radiated GBM Cell Lines Selectively Vulnerable Anti-Bcl-2 Family Agents

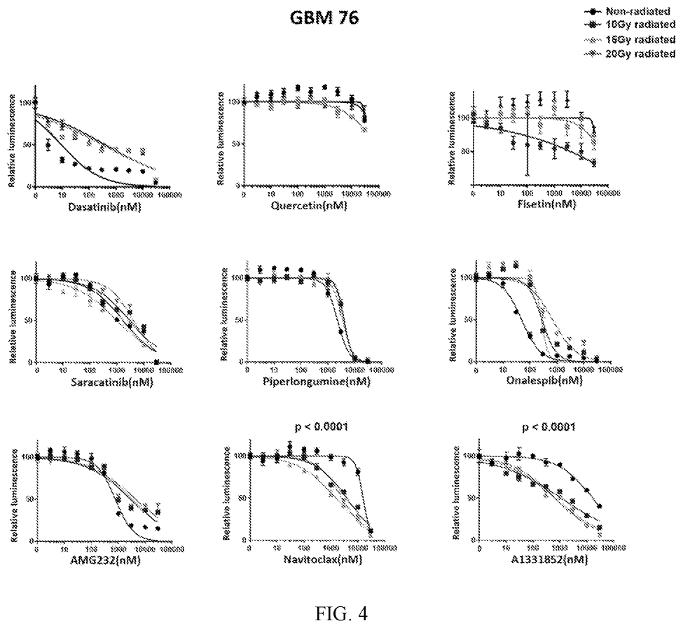

Whether prior radiation increases sensitivity to senolytic agents was investigated. The senolytic drugs tested and their associated senescent cell associated pathways (SCAPs) are detailed in Table 1. Irradiated cell lines GBM39 and GBM76 both demonstrated relatively higher sensitivity to the Bcl-2 family targeting drugs A1331852 (mean+/−SD, range for each cell line) and navitoclax (mean+/−SD, range for each cell line) than non-radiated cells (p<0.0001) (FIG. 3, FIG. 4). Both navitoclax and A1331852 target the Bcl-2 family of anti-apoptotic proteins. Specifically, navitoclax targets Bcl-2 and Bcl-XL, whereas A1331852 targets Bcl-XL only.

TABLE 1
Senescent Cell Anti-Apoptotic Pathways (SCAPS) and Inhibitors.

| Senescent Cell Anti- | |
|---|---|
| Apoptotic Pathways (SCAPS) | Inhibitors |
| 1. Bcl-2, Bcl-X_{L} | Navitoclax, A1331852, A1155463, |
| | Venetoclax |
| 2. PI3Kδ, AKT, ROS- | Quercetin, Fisetin, Piperlongumine |
| protective, metabolic | |
| 3. MDM2, p53, p21, serpine | Quercetin, Fisetin, Dasatinib |
| 4. Ephrins, dependence | Dasatinib, Piperlongumine |
| receptors, tyrosine kinases | |
| 5. HIF-1^{α} | Quercetin, Fisetin |
| 6. HSP-90 | Onalespib |

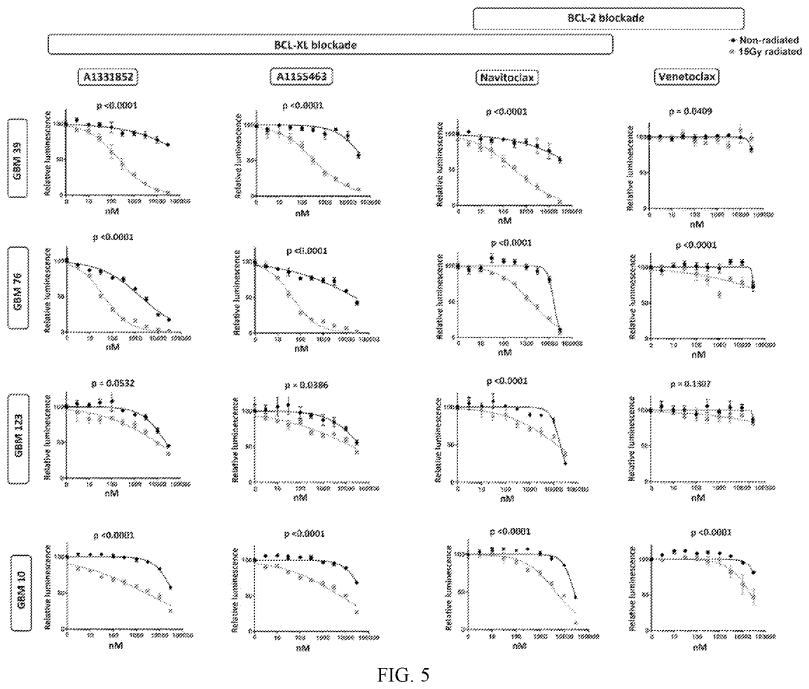

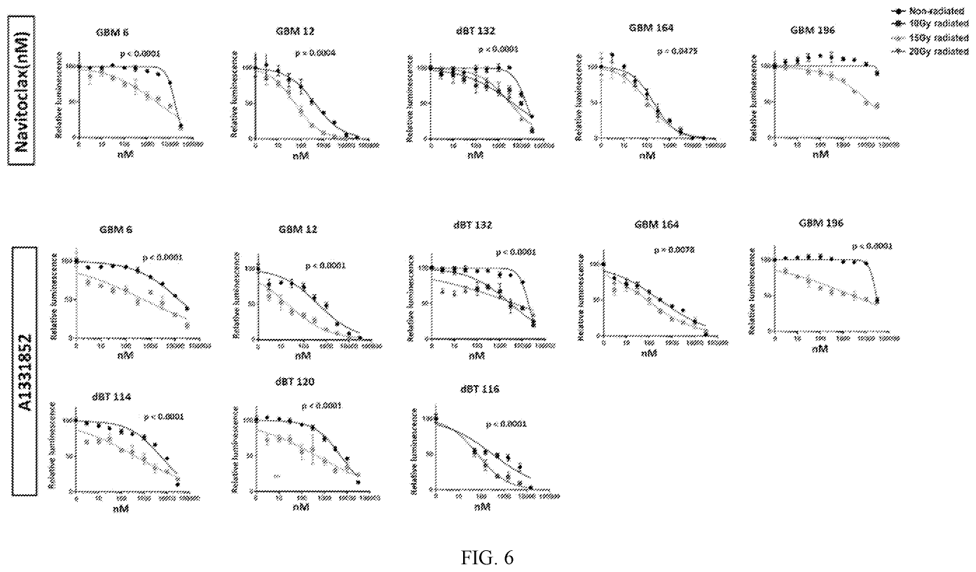

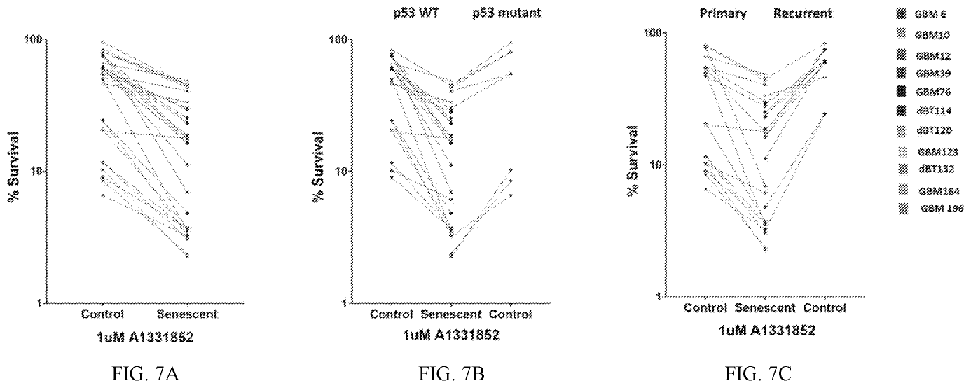

To further discriminate between the dependence of radiated human GBM upon Bcl-XL and Bcl-2, navitoclax and A1331852, as well as A1155463-a Bcl-XL-selective inhibitor, and venetoclax, a Bcl-2-selective inhibitor, were evaluated. The Ki values for each of these 4 drugs for anti-apoptotic Bcl-2 family members (Bcl-XL, BCl-2 and Bcl-W) is shown in Table 4. To date, no drug is available that selectively inhibits Bcl-W. Ablation of radiated cells was seen across all lines (GBM10, 39, 76, 123), for all drugs, except for venetoclax (FIG. 5). Three of four lines showed no selective ablation with Venetoclax; lower viability was seen with high dose Venetoclax in radiated GBM 10 cells. These data suggested Bcl-XL to be the most relevant therapeutic target for ablation of previously radiated senescent-like GBM. To further evaluate the reproducibility of Bcl-XL dependence in radiated human glioblastoma, A1331852 and/or navitoclax was tested in several additional molecularly diverse cell lines with and without prior 10, 15 or 20 Gy radiation (FIG. 6). The molecular characteristics of the cell lines utilized are summarized in Table 2. Among the 13 human GBM cell lines evaluated, meaningful analysis was possible in all with the exception of GBM43, for which insufficient cells survived radiation to permit testing. Results of the pharmacologic Bcl-XL inhibition studies performed are summarized in Table 3 and FIG. 7. While the IC₅₀ of Bcl-XL inhibition with and without radiation varied markedly between lines, radiated GBM was reproducibly more susceptible to Bcl-XL inhibition than non-radiated GBM.

TABLE 2
Characteristics of cell lines.

| | | | Recurrence | MGMT | | | | | | | | Tert | |
|---|---|---|---|---|---|---|---|---|---|---|---|---|---|
| Line | Sex | age | Status | methylation | Subtype | IDH1 | CDKN2A | PTEN | EGFR | TP53 | Met | Prom | Other |
| 6 | M | 65 | Primary | U | Clas | | L | L | A (v3) | M | A | M | |
| 10 | M | 41 | Recurrent | U | Mes | | L | LM | A | | A | M | |
| 12 | M | 68 | Primary | M | Mes | | L | | AM | ML | | M | |
| 39 | M | 51 | Primary | M | Mes | | L | LM | A (v3) | | A | M | MDM4 & |
| | | | | | | | | | | | | | PIKC32B |
| | | | | | | | | | | | | | Amp |
| 43 | M | 69 | Primary | U | Mes | | L | | | M | | M | |
| 76 | M | 38 | Recurrent | M | Clas | | L | LM | A (V3) | | A | M | |
| 123 | F | 62 | Primary | U | PN | | | L | A (V2) | M | | M | CDK4&Myc |
| | | | | | | | | | | | | | Amp; ATRX |
| | | | | | | | | | | | | | mut |
| 196 | F | 30 | Primary | M | ND | M^{hh} | L | L | A | MA | A | — | PDGFR & |
| | | | | | | | | | | | | | RB1 Amp |
| 164 | F | 38 | Primary | M | PN | M^{h} | L | L | | | A | — | PDGFR Amp; |
| | | | | | | | | | | | | | NF1 loss; |
| | | | | | | | | | | | | | ATRX trunc |
| 114* | M | 68 | Primary | M | ND | | L | L | A | | | M | |
| 116* | F | 56 | Primary | M | Mes | | L | LM | A | M | A | M | |
| 120* | M | 57 | Recurrent | U | PN | | L | L | A | M | A | M | |
| 132* | M | 75 | Primary | U | Clas | | L | L | A | M | | M | |

*= dBT (maintained in culture)
M = Mutant
A = Amplified
L = Loss
Subtypes: Classical (Clas), Mesenchymal (Mes), Proneural (P), Not determined (ND)
^{h}heterozygous R132H
^{hh}homozygous R132H

TABLE 3
Results of pharmacologic Bcl-XL inhibition studies.

| Cell lines | | | Relative | | |
|---|---|---|---|---|---|
| (passage | IC50 0f A1331852(uM) | | sensitivity | | N of |
| number) | Control | 10Gy | (RS) | P value | experiments |
| dBT132 (P10) | 18.166 | 4.311 | 4.213871 | p < 0.0001 | 1 |
| GBM76 (P2) | 23.464 ± 6.762161 | 0.999867 ± 0.36727 | 31.77018 ± 23.61 | p < 0.0001 | 3 |
| GBM6 (P4) | 14.128 | 0.5316 | 26.57637 | p < 0.0001 | 1 |
| | Control | 15Gy | RS | | |
| dBT114 (P11) | 6.3175 ± 1.0925 | 0.33825 ± 0.07205 | 20.28 ± 7.55 | p < 0.0001 | 2 |
| dBT120 (P10) | 5.179 | 0.4581 | 11.30539 | p < 0.0001 | 1 |
| GBM12 (P3) | 0.39 ± 0.07 | 0.0229 ± 0.007611 | 11.30539 ± 8.282214 | p < 0.0001 | 3 |
| GBM76 (P2) | 23.464 ± 6.762161 | 1.225467 ± 0.41632 | 20.64494 ± 5.632602 | p < 0.0001 | 3 |
| GBM6 (P4) | 14.128 | 0.5316 | 26.57637 | p < 0.0001 | 1 |
| GBM10 (P2) | 39.97 | 2.469 | 16.18874 | p < 0.0001 | 1 |
| GBM123 (P3) | 22.991 | 9.356 | 2.457354 | P = 0.0532 | 1 |
| GBM39 (P2-3) | 107.8895 ± 152.35 | 0.080547 ± 0.093 | 513.8149 ± 711.64 | p < 0.0001 | 3 |
| GBM196 (P2) | 27.336 | 2.549 | 10.72420557 | p < 0.0001 | 1 |
| GBM164 (P2) | 0.2973 | 0.07774 | 3.824286 | | |
| | Control | 20Gy | RS | | |
| dBT116 (P8) | 0.2899 | 0.06349 | 4.566073 | p < 0.0001 | 1 |
| dBT132 (P10) | 18.166 | 2.934 | 6.191547 | p < 0.0001 | 1 |
| GBM76 (P2)- | 23.464 ± 6.762161 | 0.679 ± 0.049209 | 34.24256 ± 7.988135 | p < 0.0001 | 3 |
| GBM6 (P4)- | 14.128 | 0.8452 | 16.71557 | p < 0.0001 | 1 |
| GBM39 (P3) | 1.30465 ± 1.01435 | 0.071005 ± 0.010255 | 16.65831 ± 11.87971 | p < 0.0001 | 2 |
| | Control | TMZ | RS | | |
| GBM76 (P3)- | 1.685 | 0.05973 | 28.21028 | p < 0.0001 | 1 |
| GBM39 (P2-3) | 1.7164 ± 2.182154 | 0.041846 ± 2.182154 | 41.01706 ± 2.182154 | p < 0.0001 | 3 |

TABLE 4
Ki values for Bcl-XL, BCl-2 and Bcl-W.

| | BCL-XL | BCL-2 | BCL-W | |
|---|---|---|---|---|
| Inhibitors | Ki (nM) | Ki (nM) | Ki (nM) | Comment |
| Navitoclax | 0.055 | 0.044 | 21 | BCL-XL, BCL-2 |
| | | | | dual inhibitor |
| A1331852 | <0.010 | 6 | 4 | BCL-XL specific |
| | | | | inhibitor |
| A1155463 | <0.010 | 74 | 8 | BCL-XL specific |
| | | | | inhibitor |
| Venetoclax | 48 | <0.010 | 245 | BCL-2 specific |
| | | | | inhibitor |

The inhibition constant, Ki, denotes the equilibrium constant--the concentration of drug needed to occupy half the free protein. Lower Ki indicates higher affinity.

### Time-Point of Bcl-XL Inhibitor Sensitivity

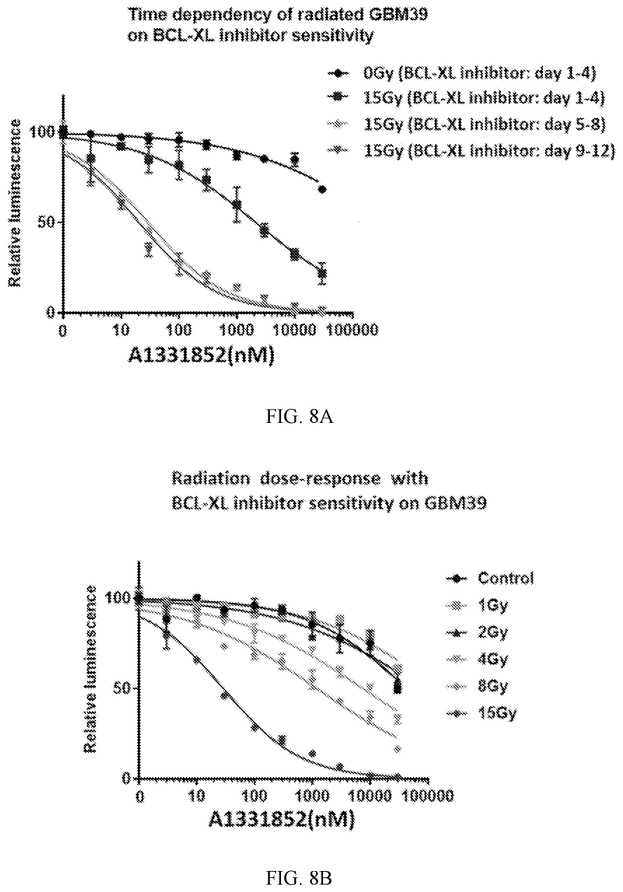

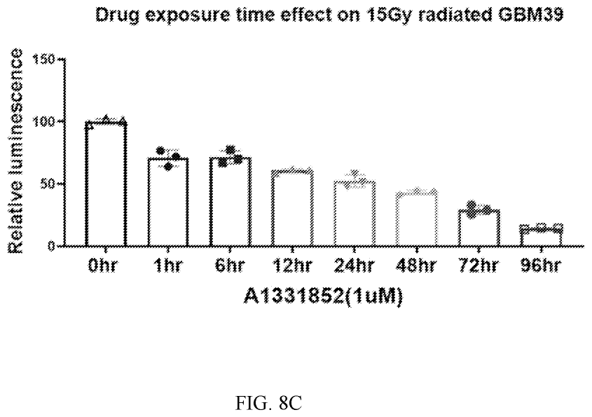

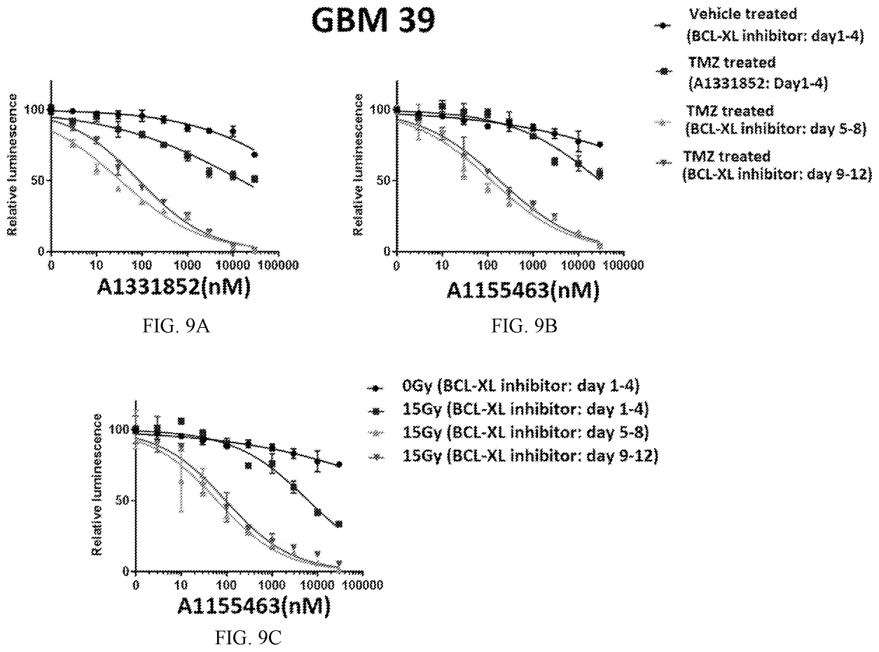

The previous experiments maintained radiated cells for 2-4 weeks after radiation prior to senolytic drug testing. While the senescence process often takes weeks, the apoptotic pathways regulated by Bcl-2 family members are dynamically regulated within days following DNA damage. As such, GBM39 was used to ask if a minimal period of time after radiation must elapse following radiation prior to onset of Bcl-XL sensitivity. Using a timed assay following radiation, GBM39 was treated with Bcl-XL inhibitors A1331852 or A1155463 for 4 days, starting 1, 5, or 9 days after radiation. Analysis was performed at the end of the 4 days drug exposure. While efficacy was seen upon treatment starting one day after radiation, more complete cell ablation was observed with a leftward shift of the dose response curve, four days elapsed after radiation prior to starting treatment 5 or 9 days following radiation (FIGS. 8-9).

### Dependency on Drug Exposure Time and Radiation Dose

Whether a minimal radiation dose was required to induce susceptibility cell death with Bcl-XL inhibition was investigated. Radiation doses of 4 Gy or higher in GBM39 promoted sensitivity to A1331852 with increasing efficacy up to 15 Gy (FIG. 8B). For most cell lines, radiation doses of 10-20 Gy yielded similar results (FIG. 6 and Table 3). The duration of continuous Bcl-XL inhibitor exposure required to observe senolytic effect was investigated. To accomplish this, GBM39 was exposed to drug for varying durations from 0-96 hours, with drug then washed off and cells maintained in normal growth media thereafter until time of analysis. Although some most impact was observed even with 1 hour of treatment, maximal impact was seen in cells that received sustained exposure to Bcl-XL inhibition for 96 hours.

### Elimination of TMZ Treated Senescent Glioma

Radiation and TMZ are both routinely administered to patients with GBM. Both may induce senescence and modulate apoptotic machinery. To determine whether TMZ (100 μM) exposure induces selective susceptibility to Bcl-XL inhibition, the GBM cell lines were pre-treated with TMZ for 20 days and then analyzed various anti-Bcl-family agents. Using GBM76, and GBM39, it was found that prior 20 days TMZ exposure induced sensitivity to the Bcl-xL inhibition, but not BCL-2 inhibition as previously observed following radiation (FIG. 8-9).

Based on the selectivity of A1331852 and A1155463 to Bcl-XL and venetoclax to Bcl-2 (Table 4), the impact of siRNA constructs that knockdown Bcl-2 family members and scrambled controls on survival in radiated and non-radiate GBM was evaluated. Compared to scrambled controls, Bcl-2 and Bcl-W knockdown elicited no impact on cell survival whereas Bcl-XL knockdown significantly decreased survival of radiated cells.

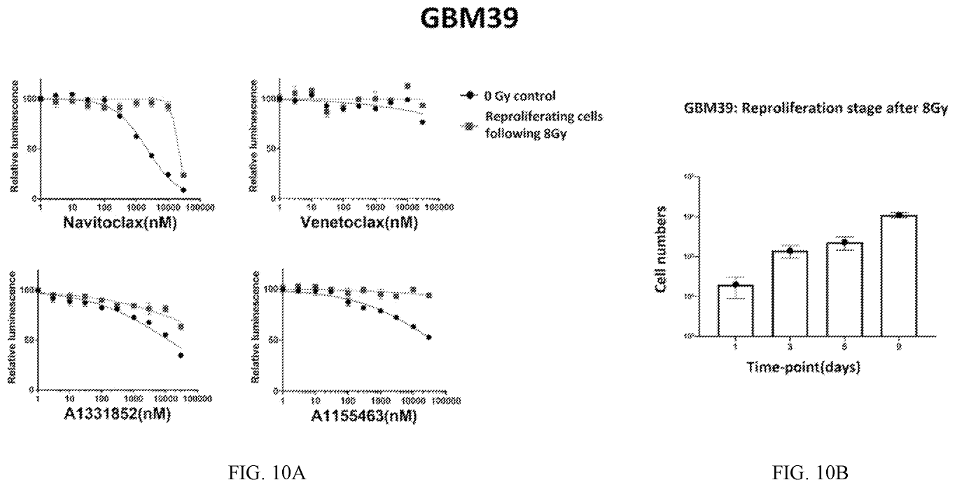

Finally, whether prior cytotoxic therapy would be sufficient to permanently induce sensitivity to Bcl-XL blockade, or if cells must remain in a senescent-like non-proliferative state to maintain sensitivity was investigated. In one culture maintained over 6 weeks following 8 Gy radiation, proliferative activity resumed, with a doubling time ultimately unchanged from the parent culture. Cells in the culture that “escaped senescence” to resume proliferative behavior lost sensitivity to Bcl-XL inhibition (FIG. 10). This result is consistent with the observation that gliomas derived from both primary and recurrent lesions (FIG. 6) proved relatively insensitive to Bcl-XL inhibition until placed into a state of proliferative arrest with either TMZ or radiation.

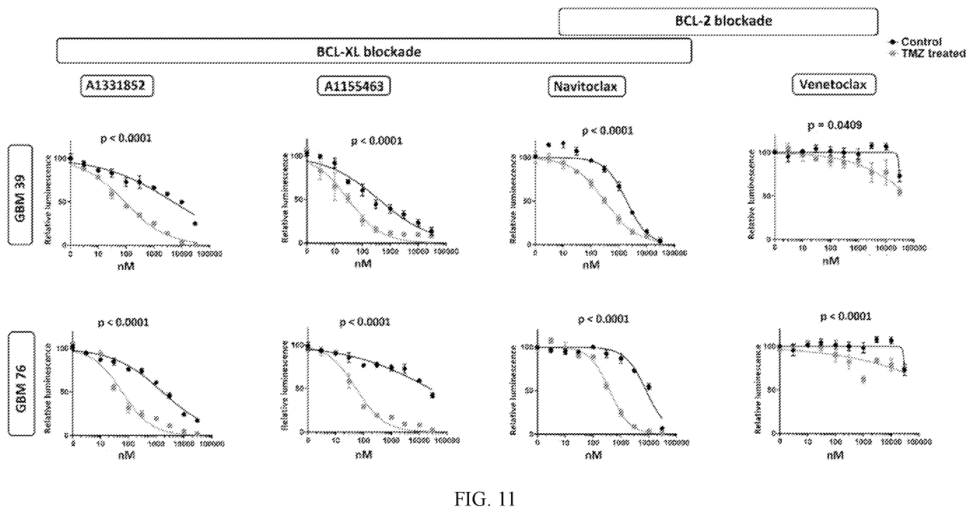

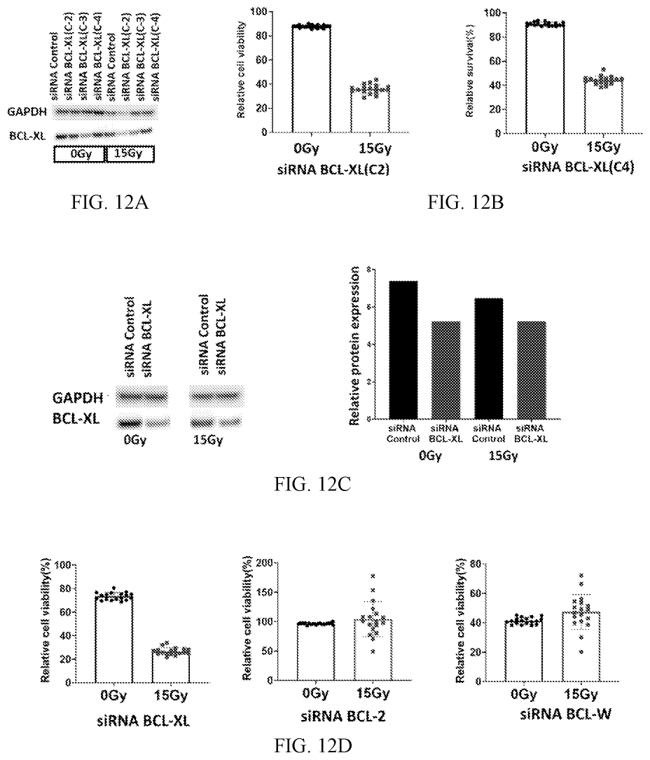

FIG. 11 and FIG. 12 show additional information related to Bcl-XL inhibition and radiated GBM vulnerability to Bcl-XL knockdown, respectively.

### Example 2: Bcl-XL-Dependent Senescent-Like Cells, Inhibitors Thereof; and Glioma Recurrence

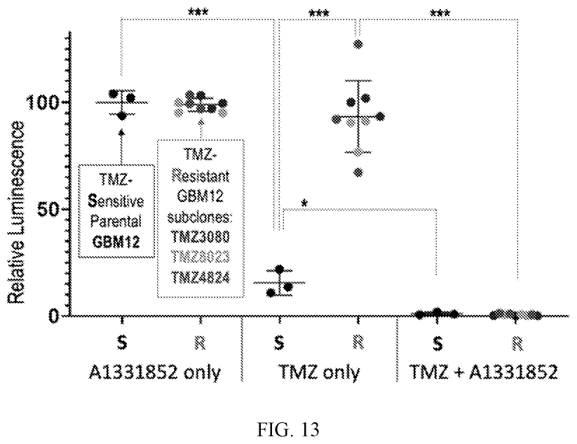

GBM12 parental line, and 3 independent TMZ-resistant subclones were exposed to 10 μM TMZ in addition to 1 μM A1221852 for 5 days prior to evaluation of cell viability by CellTiter-Glo. Multiple independent TMZ-resistant GBM subclones were robustly induced to Bcl-XL dependency upon TMZ re-challenge, despite differing mechanisms of acquired TMZ resistance (FIG. 13). A1331852 increased TMA-induced ablation in parental TMZ-sensitive cells (S), but also induced complete ablation in 3 separate TMZ-resistant subclones (R).

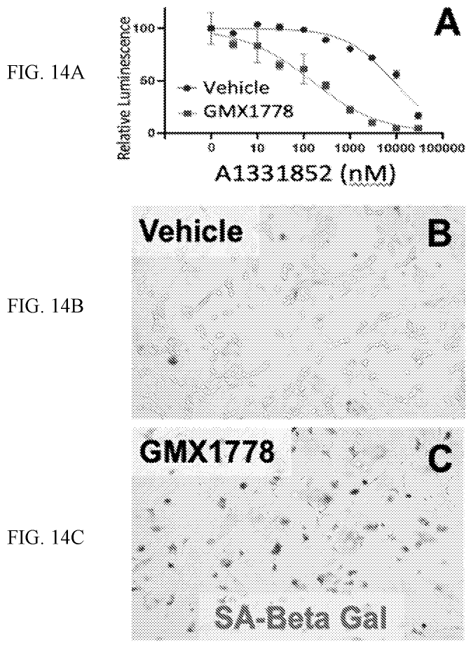

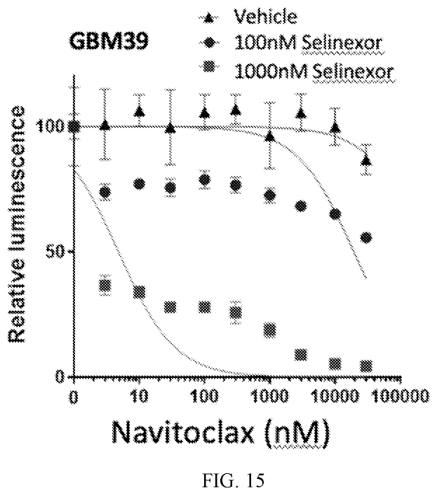

Samples from 10 pre-recurrent human glioma patients who underwent radiotherapy were used to determine whether baseline Bcl-XL dependency of such non-mitotic human glioma is further augmented by ex vivo exposure to TMZ, MDM2 inhibition, or other candidates found to induce glioma senescence, including inhibitors of NAMPT such as GMX1778 (FIG. 14), XPO1 (FIG. 15) or CKD4/6. In FIG. 14, non-radiated GCM164 cells were exposed to the NAMPT inhibitor GMX1778 at 50 nM for 10 days, which induced a Bcl-XL dependent state, with reduced A1331852 IG50 (p<0.0001). In addition, there was increased SA-beta-gal activity, which is consistent with a senescent-like phenotype. In FIG. 15, non-radiated GBM39 cells were exposed to Selinexor at 100 nM or 1000 nM with concentrations of navitoclax ranging from 1 nM to 30000 nM. Selinexor exposure sensitized non-radiated GBM39 cells to navitoclax in a dose-dependent manner. Error bars show SEM of technical replicates (p>0.0001). Parallel samples from the OR are subject to phenotypic evaluation via CyTOF and SN-RNAseq, and are compared to samples from non-senescent primary or recurrent glioblastoma specimens to identify phenotypic signatures or specific biomarkers that discriminate senescent from non-senescent human glioma.

### Example 3: Increasing Extent of Ablation—a Senolytic Approach to Promote Apoptosis of Latent Glioblastoma Following Chemoradiation

### Methods:

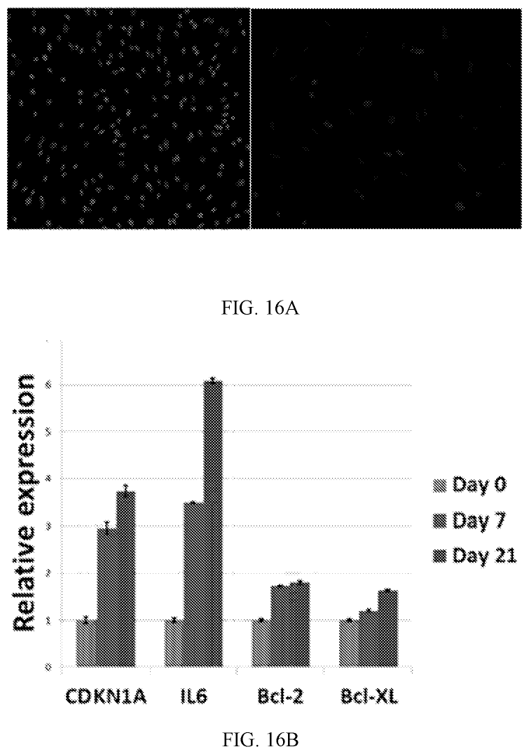

Senolytic drugs were screened using human glioblastoma cells radiated with 10-20 Gy, or 100 uM TMZ and then maintained for 3 to 4 weeks. Senescence was confirmed via beta-galactosidase staining, elevated p21 transcript and loss of nuclear laminin B1 immunohistochemistry (FIGS. 16A-16B). IC₅₀ was compared in radiated (senescent) vs non-radiated cells using the cell-titer glow assay. 1 month after in vivo radiation of intracranial tumors, convection-enhanced delivery (CED) of drug was performed. Any change in tumor bioluminescence was evaluated 1 month thereafter. CED infusion efficacy was confirmed via MRI.

### Results:

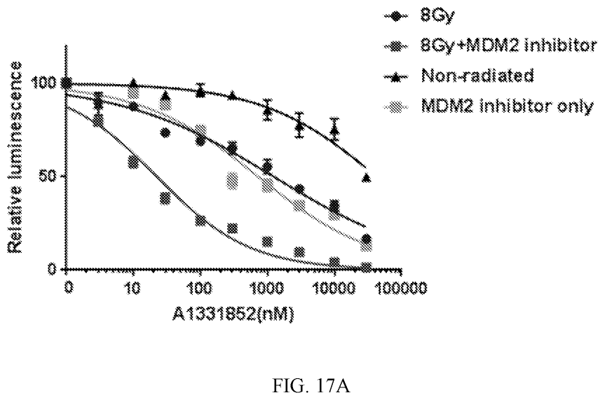

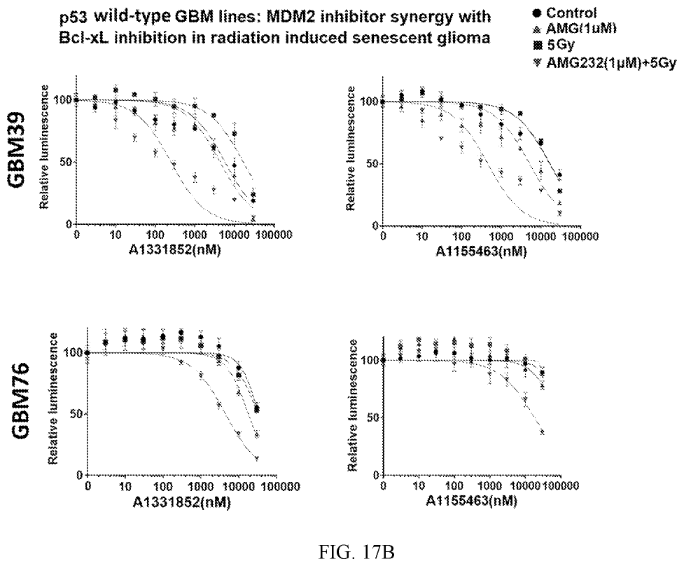

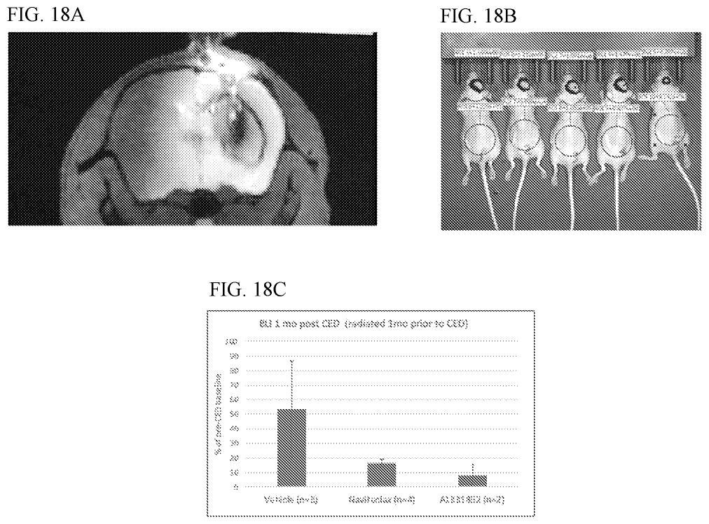

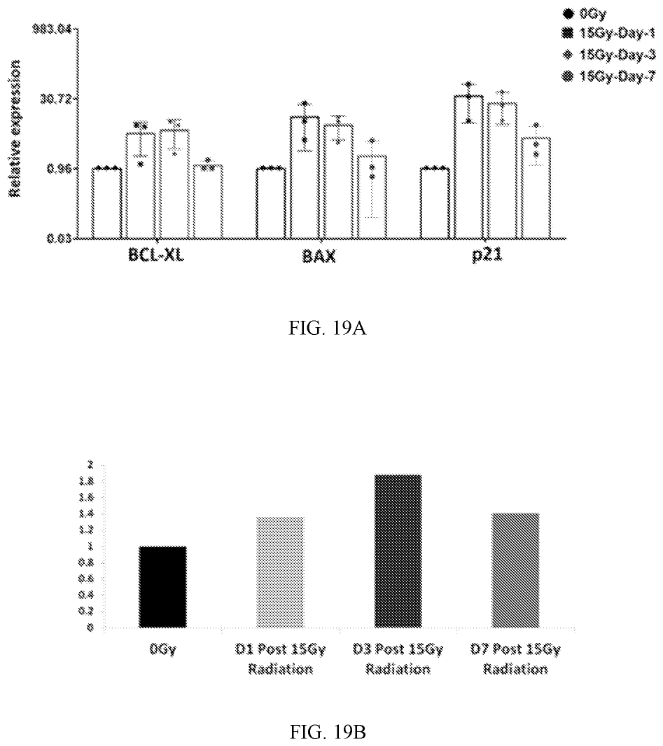

Of the senolytic drugs screened, only Bcl-xL inhibitors (A1331852, A1155463 and navitoclax, see Table 5) but not BCL-2-selective nor other inhibitors showed senolytic effect (lower IC₅₀) in cells surviving TMZ or radiation. Findings were consistent in all IDH-WT lines tested, including p53-mutant and WT lines. Administration of AMG232 (MDM2 inhibitor) prior to radiation further increased senolytic efficacy (IC₅₀<12 nM, compared to >20,000 nM in naïve cells; FIG. 17A). Convection-enhanced delivery (CED) was performed to infuse senolytic drugs into orthotopic tumors 1 month following radiation. By 1 further month thereafter, tumor bioluminescence had decreased by 84.8% (navitoclax) or 93.1% (A1331852), compared to vehicle (39.7%; overall p=0.02). Results for navitoclax may have been hampered by poor drug diffusion, since separate pilot mass spectrometry data suggest poor Navitoclax diffusion after CED (10-fold decrease within 0.5 mm). Additionally, there was a senolytic effect of navitoclax or A1331852 in radiated orthotopic glioma (FIGS. 18A-18C).

TABLE 5
Senescent Cell Anti-Apoptotic Pathways (SCAPS) and Inhibitors

| Senescent Cell Anti- | |
|---|---|
| Apoptotic Pathways (SCAPS) | Inhibitors |
| 1. Bcl-2, Bcl-X_{L} | Navitoclax, Fisetin, A1331852, A1155463 |
| 2. PI3Kδ, AKT, ROS- | Quercetin, Fisetin, Piperlongumine |
| protective, metabolic | |
| 3. MDM2, p53, p21, serpine | Quercetin, Fisetin, Dasatinib |
| 4. Ephrins, dependence | Dasatinib, Piperlongumine |
| receptors, tyrosine kinases | |
| 5. HIF-1α | Quercetin, Fisetin |
| 6. HSP-90 | 17-AAG, Onalespib Geldanamycin, |

### Example 4: Therapy to Target Pre-Recurrent Glioma

Therapy-resistant residual cells persist in a latent state a long time before inevitable recurrence. Conventional radiation and TMZ treatment cause oxidative stress and DNA damage resulting senescent-like state of cell-cycle arrest. However, increasing evidence demonstrates escaping senescence leads to tumor recurrence. Thus, the ablation of senescent tumor cells after chemo-radiation may be an avenue to limit tumor recurrence.

### Methods:

100 μM TMZ for 7 days or 10-20 Gy radiation (cesium gamma radiator) was used for senescence induction in human glioblastoma in vitro and senescence was confirmed by SA-Beta gal staining and RT-PCR, and protein level. Replication arrest was assessed by automated quantification of cellular confluence (Thermo Scientific Series 8000 WJ Incubator). The IC₅₀ for several senolytics targeting multiple SCAPs, including dasatinib, quercetin, AMG-232, fisetin, onalespib, navitoclax, and A1331852, was evaluated in senescent vs. proliferating cells.

### Results:

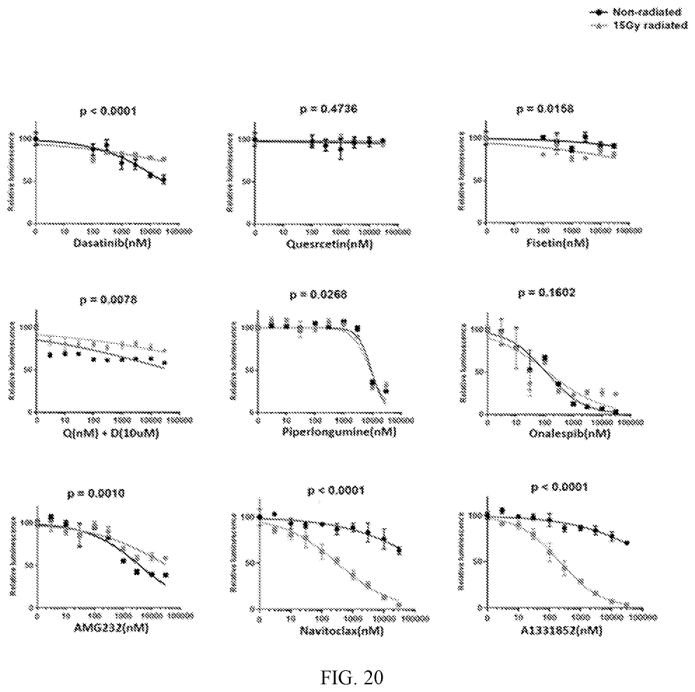

Among the senolytics tested, the Bcl-XL inhibitors A1331852 and navitoclax both shown senolytic effect by selectively killing radiated, senescent tumor cells at lower concentrations as compared to 0 Gy treated non-senescent cells (FIG. 20). Across 12 GBM cell lines, IC₅₀ for senescent cells was 6-500 times lower than non-senescent GBM (p<0.005). Such differential sensitivity to Bcl-XL inhibition after radiation has also observed by Bcl-XL siRNA knockdown in radiated glioma.

### Example 5: MDM2 Inhibition Augments Bcl-xL-Mediated Senolytic Ablation Inhibition for p53 Wild Type Glioblastoma

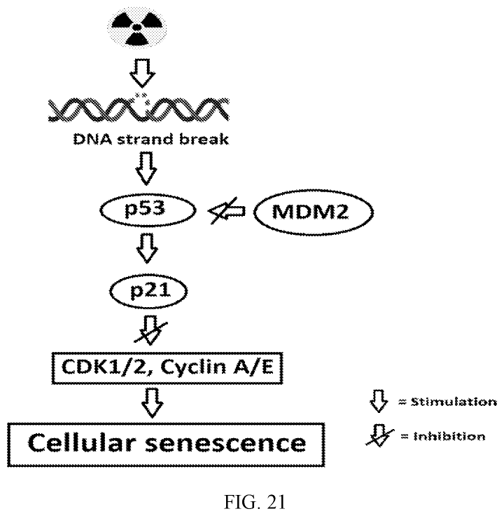

Bcl-XL inhibition promotes cell death in senescent GBM cells. Cell death may be further augmented by MDM2 inhibition in p53-WT cells as MDM2 is a negative regulator of the p53-p21 pathway following radiation-induced DNA breakdown (FIG. 21).

### Methods:

P53-mutant (GBM6/GBM123) and p53 WT (GBM39/GBM76) human GBM cells were treated with 5 Gy radiation or TMZ (100 μM) following 48 hours of MDM2 inhibitor (AMG232, 1 μM) or vehicle. Cells were then maintained for seven days, to allow time for cells to establish a senescent-like phenotype. Cells were then incubated with a Bcl-XL inhibitor (A1331852 or A1155463) in each group to determine if MDM2-inhibitor pre-treatment could help sensitize cells to senolytic ablation via Bcl-XL inhibition. Cell viability was evaluated after 5 days using the CellTiter-Glo luminescence-based cell viability assay.

### Results:

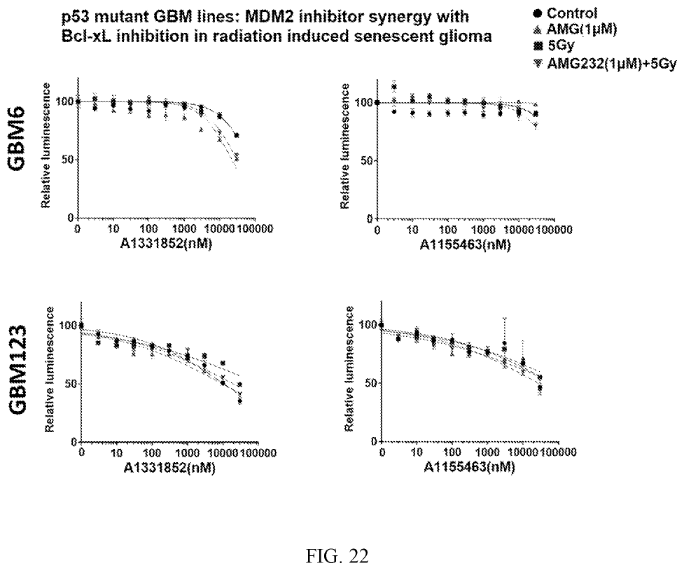

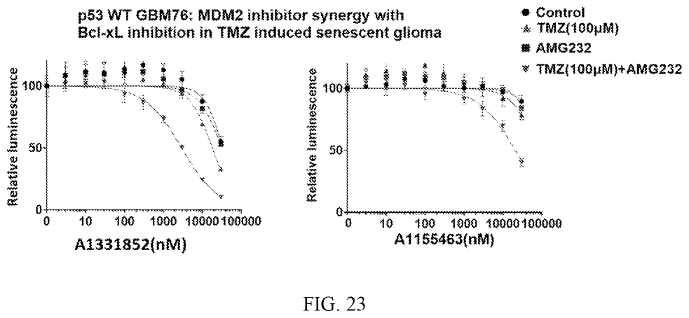

MDM2 inhibitor treatment prior to radiation increased the expression of p21 in p53-WT but not p53-mutant cells, consistent with more intense induction of senescence. Low dose radiation (5 Gy) is sufficient to induce senescence or Bcl-XL sensitivity. Here, MDM2 pre-treated radiated cells showed significantly increased vulnerability to A1331852- or A1155463-induced cell death compared to cells treated with radiation alone (FIG. 17B). In GBM76, addition of MDM2 prior to radiation decreased the IC₅₀ of A1331852 from 18.1 μM to 4.5 μM (p=0.0036). In GBM39, addition of MDM2 prior to radiation decreased the IC₅₀ of A1331852 from 5.6 μM to 0.3 μM (p=0.0003). MDM2 inhibition also increased sensitivity of GBM76 and GBM39 to Bcl-XL inhibition when used prior to TMZ treatment, suggesting a general ability to promote senescence after cytotoxic stress. By contrast, MDM2 failed to promote sensitivity to senolytic ablation in p53-mutant cell lines GBM6 and GBM123 (FIGS. 22-23; p>0.05).

### Example 6: Bcl-XL Inhibits PUMA-Mediated Glioblastoma Cell Death after Radiation

This Example demonstrates that BCL-XL can mediate GBM survival following radiation by preventing PUMA from activating BAX-mediated apoptosis.

### Experimental Procedures:

### Cell Culture and Growth Conditions:

GBM6, GBM39, and GBM164 human glioblastoma cell lines were obtained from the National (Patient-derived Xenografts) PDX resource. All cell lines were cultured and maintained in antibiotic-free FBS media, composed of (10% Fetal Bovine Serum (FBS) and 90% Dulbecco's Modified Eagle's medium (DMEM)). Cells were passaged regularly once they reached 70-90% confluence using phosphate-buffered saline (PBS) and 1× Trypsin-EDTA (0.05% Trypsin and 0.53 mM EDTA). All experiments were performed on these cells at the 3rd cellular passage.

### Irradiation of Cells:

1-3×10⁶ GBM cells were plated either in 10 cm or 7 cm sterile tissue culture vessels and left in culture for 2-3 days till they become 50-60% confluent then exposed to different doses of radiation (1, 2, 4, 8, 10, and 15Gy) using cesium gamma irradiator.

### Cell Viability Assay:

The number of viable cells in culture was measured using the CellTiter-Glo® Luminescent Cell Viability Assay as per the manufacturer's instructions and protocol (Promega, G7570, G7571, G7572, and G7573). The CellTiter-Glo® Luminescent Cell Viability Assay is a homogeneous method to determine the number of viable cells in culture based on quantitation of the ATP present, which signals the presence of metabolically active cells.

### Caspase-3 Assay:

Caspase-3 enzymatic activity was measured in cell lysates prepared from GBM cell lines using The Caspase-3 Activity Assay Kit (Cell Signaling Technology, #5723) as per the manufacturer's instructions and protocol. The Caspase-3 Activity Assay Kit is a fluorescent assay that detects the activity of caspase-3 in cell lysates. It contains a fluorogenic substrate (N-Acetyl-Asp-Glu-Val-Asp-7-amino-4-methylcoumarin or Ac-DEVD-AMC) for caspase-3. During the assay, activated caspase-3 cleaves this substrate between DEVD and AMC, generating highly fluorescent AMC that can be detected using a fluorescence reader with excitation at 380 nm and emission between 420-460 nm. Cleavage of the substrate only occurs in lysates of apoptotic cells; therefore, the amount of AMC produced is proportional to the number of apoptotic cells in the sample.

Quantitative Real-Time PCR (qRT-PCR):

RNA was extracted from GBM cells 24 hours after radiation. Briefly, cells were washed with PBS before being homogenized with TriZol reagent (Invitrogen). RNA precipitation was performed at −20 C overnight (12-18 hours). The precipitated RNA pellets were dissolved in RNase-free water and concentration was measured by absorbance at 260 nm (A260) using Nanodrop2000. cDNA synthesis was performed with 1 μg of total RNA using M-MLV reverse transcriptase kit (ThermoFisher, #28025013 and 28025021) as per the manufacturer's protocol. 25 ng of cDNA was used for real-time PCR with Taqman gene expression assay targeting different BCL-2 family members on ABI 7500/7500-Fast Real-Time PCR System (Applied Bioscience). The relative expression of each gene was determined by the ΔΔCT method.

### Protein Analysis by Western Blotting:

Cells grown in six-well plates, 10 cm dishes, or T-25 flasks were washed with phosphate-buffered saline (PBS), dissociated in 1× Trypsin-EDTA (0.05% Trypsin and 0.53 mM EDTA), and collected in 1.5 micro-centrifuge tubes as a cell pellet. The cell pellet was then lysed using lysis buffer (10% RIPA lysis buffer, 4% Protease-Inhibitor cocktail, 1% Phosphatase-Inhibitor cocktail-2, 1% Phosphatase-Inhibitor cocktail-3, and 84% Molecular grade water). The cell palate with the lysis buffer was then sonicated for 30 minutes in a water bath sonicator (one-minute sonication every other minute for a total of 30 minutes). The whole lysate was centrifuged for 10 minutes at the speed of 17,000 g to collect the supernatant as the final protein lysate. The concentration of the final protein lysate was then measured using the BCA kit and albumin as a standard protein (ThermoFisher, #23225). Proteins extracted from cells using lysis buffer were separated in SDS-polyacrylamide gel electrophoresis along with the protein ladder (Life Technology) using 4-12% Bis-Tris gel (ThermoFisher). Proteins were then transferred to a polyvinylidene difluoride (PVDF) membrane (Bio-Rad). The membrane was blocked in 5% fat-free milk (Cell-signaling technology) for 30 minutes, washed three times (5 minutes each) using Tris-buffered saline with tween20 (TBST), and probed with different antibodies (Cell-signaling technology).

### Co-Immunoprecipitation (Co-IP) Analysis:

Isolation of individual proteins or protein-protein complexes was performed using the Capturem IP & Co-IP Kit (Clontech-TaKaRa-cellartis, #635721) as per the manufacturer's protocol and Instructions. Briefly, 3×10\hat{ } (1×) of irradiated cells or 12×10⁶ (4×) of non-irradiated cells were used to isolate an individual protein or an individual protein complex. The same number of cells was used in all experiments (unless mentioned otherwise). Cells were counted using an automated cell counter (Life Technologies) after being washed with phosphate-buffered saline (PBS), dissociated in 1× Trypsin-EDTA (0.05% Trypsin and 0.53 mM EDTA), suspended in PBS, stained with Trypan blue dye, and mounted on Countess™ Cell Counting Chamber Slides (Invitrogen, #C10228, C10312, C10313, C10314, and C10315). The cells then were collected in 1.5 micro-centrifuge tubes as a cell pellet. The cell pellet was then lysed using lysis buffer (1% of the 100× Protease-Inhibitor cocktail provided in the kit, 1% Phosphatase-Inhibitor (100×) cocktail-2 (not provided in the kit), 1% Phosphatase-Inhibitor (100×) cocktail-3 (not provided in the kit), and 97% of the lysis buffer provided in the kit). The cell palate with the lysis buffer was then sonicated for 30 minutes in a water bath sonicator (one-minute sonication every other minute for a total of 30 minutes). The whole lysate was centrifuged for 10 minutes at the speed of 17,000 g to collect the supernatant as the final protein lysate. The concentration of the final protein lysate was then measured using the BCA kit and albumin as a standard protein (ThermoFisher, #23225) to make sure that an equal number of cells yields in equal protein concentrations since different GBM cell lines were used and the cellular size may vary between different lines, then concentrations were equalized as needed. A 100 μL of cell lysate was used to isolate a single protein or a protein complex, incubated with the specific antibody overnight at 4 C on a shaker, and run through an isolation column provided with the kit as per the manufacture's protocol. The final eluted protein samples were analyzed using western blotting protocol as described above.

Viral Packaging and Gene Knock-Down by shRNA:

Plasmid sequences for shRNAs were either designed manually using commercially available soft wares or purchased as already designed sequences from Addgene. The sequence was provided in the form of a Bacterial plasmid. Bacteria were cultured in Lysogeny broth (LB) media and Ampicillin (to kill any intervening bacteria that do not have the plasmid of interest). A single bacterial colony was selected from the bacterial culture and further expanded. DNA Plasmid from the bacteria was isolated and purified using PureLink™ HiPure Plasmid Miniprep Kit (Invitrogen, #K210002 and K210003) as per the manufacturer's protocol and Instructions. Isolated DNA concentration and purification were measured using the NanoDrop. Viral packaging was performed as per the manufacturer's (Addgene) protocol and Instructions using 10 ug of DNA plasmid to transfect 3.8×10⁶ HEK293 cells in a 10 cm tissue culture dish. The virus was harvested 72 hours post-transfection. Viral supernatant was centrifuged at 500 g for 5 minutes to pellet any packaging cells that were collected during harvesting. The supernatant was filtered through a 0.45 um polyethersulfone (PES) filter. Viral supernatant was aliquotted and snap-frozen in liquid nitrogen and stored at −80 C to avoid loss of titer till the time transduction was performed. 5-8×10⁵ GBM cells were plated in 6-well plates and incubated at 37 C overnight (16-18 hours). The following day, 10-20 μL of viral supernatant were added to each well and incubated at 37 C for 36-72 hours. The protein then was extracted to confirm the knock-down and cells were re-plated for any further experiments.

### Gene Knock-Out by CRISPR Cas9 (Lentiviral Method for Single-Gene Knock-Out):

Plasmids encoding for Cas9 protein were purchased from Addgene online service along with plasmids encoding for Puromycin-resistance gene. Provided bacterial plasmids were cultured in Lysogeny broth (LB) media and Ampicillin. A single bacterial colony was selected from the bacterial culture and further expanded. DNA Plasmid from the bacteria was isolated and purified using PureLink™ HiPure Plasmid Miniprep Kit (Invitrogen, #K210002 and K210003) as per the manufacturer's protocol and Instructions. Isolated DNA concentration and purification were measured using the NanoDrop. Viral packaging was performed as mentioned earlier. GBM cells were cultured in a 6-well plate at a density of 5-8×10⁵ cells in 2 mL of Antibiotic-free media per well. Cells were incubated overnight (16-18 hours) at 37 C. The following day, 10-20 μL of viral supernatant were added to each well and incubated at 37 C for 48-72 hours. Transduced cells then were treated with Puromycin for 48 hours to ablate all non-transduced cells. Survived cells were expanded in culture. The presence of Cas9 in the cells was verified by western blot using the Guide-it Cas9 Polyclonal Antibody (TaKaRa, Cat. Nos. 632606 & 632607). Other viral vectors encoding for other genes (PUMA, BCL-XL, and BAX), were generated using the same way as described above. Cas9 gene editing activity and the success of CRISPR knock-out were firstly checked using the Guide-it Mutation Detection Kit (TaKaRa, Cat. No. 631448). Then another step of confirmation by western blotting analysis was performed as shown in the figures using specific antibodies to each knocked-out gene. Quantitative real-time PCR (qRT-PCR) also was used to confirm the knock-out as described earlier, knocked-out genes showed undetermined results in results even with high RNA concentrations confirming the success of CRISPR/Cas9 knock-out using this method.

### Gene Knock-Out by CRISPR Cas9 (Gesicle Production System Method for Multiple Genes Knock-Out):

Gesicle Production System is a novel method that can be used for direct delivery of the Cas9/sgRNA ribonucleoprotein (RNP) complex, obtaining levels of genome editing that are similar to those of plasmid-based delivery, with the added benefit of fewer off-target effects due to the short lifespan of the Cas9 protein in the cell without causing any cellular toxicity. With these benefits of the minimum off-target and cellular toxicity effects due to the short life span of the used Cas9 proteins, it was an ideal method for us to use to deliver multiple sgRNAs targeting multiple genes. Briefly, it's a method to deliver Cas9/sgRNA Riboneucloprotein (RNP) complexes using cell-derived nanovesicles called gesicles. Gesicles are vesicles released from the plasma membrane of mammalian producer cells which can carry any cargo, such as proteins. CRISPR/Cas9 Gesicles are generated by the co-expression of Cas9 protein, a customer-designed sgRNA, and other proteins that stimulate gesicles to be released from the producer cell membrane. Once gesicles have been made, they can be harvested, concentrated, and applied to target cells, where the active Cas9/sgRNA complex is transported to the nucleus for efficient gene editing. Reagents used in this system were purchased from Clontech-TaKaRa-cellartis (Cat. No. #632616, #632617, #632613, and #632612) and gesicle production and transfection were performed as per the manufacturer's protocol and Instructions. Briefly, sgRNAs for each gene target were designed manually and the oligos corresponding to the target-specific sgRNA designed above are annealed to form a DNA duplex and then cloned into the provided linearized delivery plasmid, pGuide-it-sgRNA1. The cloned plasmid is then diluted with dH2O and added to the Gesicle Packaging Mixes. Following a 10 minute incubation, the mix was applied to HEK293 producer cells in the presence of the A/C Heterodimerizer ligand. 48-72 hours later, gesicles containing active Cas9/sgRNA complexes are collected from the medium and concentrated via overnight centrifugation. The presence of Cas9 in the gesicles was verified by western blot using the Guide-it Cas9 Polyclonal Antibody (TaKaRa, Cat. Nos. 632606 & 632607). Cas9/sgRNA gesicles are applied to the target cells in the presence of protamine sulfate, followed by a 30 min centrifugation step to enhance gesicle-to-cell contact. Cas9 gene editing activity was checked using the Guide-it Mutation Detection Kit (Cat. No. 631448), quantitative real-time PCR (qRT-PCR), and western blotting.

### Statistical Analysis:

All the experiments were repeated at least three times. Statistical analysis was performed with one-way ANOVA and unpaired 2-tailed t-test using GraphPad prism 8.4.2. Error bars represent the SD values and significance was taken as p*<0.05, p**<0.01, p***<0.001, p****<0.0001. Unless otherwise stated, each data point reflects the average of three technical replicates. Error bars show standard deviation of biological replicates.

### Densitometric Analysis of Protein Bands:

The analysis was performed using the ImageJ software as outlined in the ImageJ documentation.

### Reagents and Antibodies:

All antibodies were purchased from Cell Signaling Technology (CST), except for the Guide-it Cas9 Polyclonal Antibody was purchased from TaKaRa, Cat. Nos. 632606 & 632607. Taqman primers were designed and purchased from ThermoFischer. Gesicle-Producer HEK293 cells were purchased from TaKaRa, while GBM6, GBM39, and GBM164 human glioblastoma cell lines were obtained from the National (Patient-derived Xenografts) PDX resource. Plasmids were provided by the Addgene online service. Reagents for the Gesicle Producer System were purchased from Clontech-TaKaRa-cellartis Inc.

### Results

Ionizing Radiation (IR) Increases PUMA mRNA and Protein Expression in Human GBM Cells

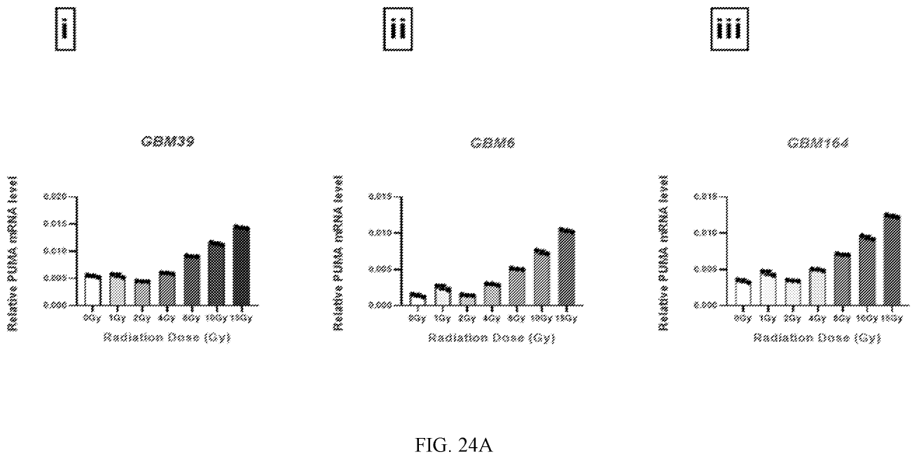

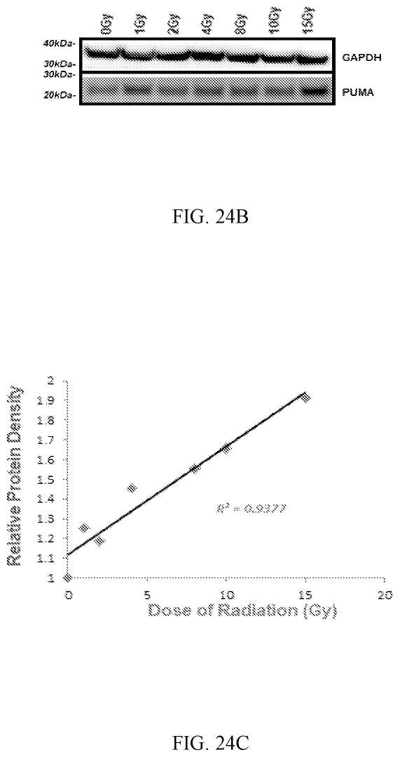

PUMA is a pro-apoptotic BH3-only member of the BCL-2 family. As implied by its name, PUMA is regulated by P53 which is activated by radiation. However, PUMA is also subject to P53-independent regulation. To determine the impact of radiation on PUMA expression, three molecularly contrasting human GBM cultures (Table 6) derived from short-term xenograft explants, including P53-WT IDH-WT (GBM39), P53-mutant IDH-WT (GBM 6), and P53-WT IDH-mutant (GBM164) lines were evaluated 24 hours following radiation (FIG. 24A). The baseline transcriptional activity of PUMA relative to GAPDH was relatively low in GBM6, perhaps reflecting the baseline lack of functional p53 activity. Nevertheless, PUMA was robustly upregulated by radiation in all three cell lines. Protein levels of PUMA were also upregulated in a dose-dependent manner with maximal PUMA abundance after 15Gy single fraction radiation (FIGS. 24B, 24C).

TABLE 6
Genetic and molecular characteristics of 3 different
GBM cell lines (GBM6, GBM39, and GBM164).

| Cell Line | 6 | 39 | 164 |
|---|---|---|---|
| Sex | Male | Male | Female |
| Age | 65 | 51 | 38 |
| Recurrence | Primary | Primary | Primary |
| Status | | | |
| MGMT | Unmethylated | Methylated | Methylated |
| Methylation | | | |
| Sub-type | Classical | Mesenchymal | Proneural |
| IDH-1 | — | — | Heterozygous |
| | | | R132H |
| CDKN2A | Homozygous | Heterozygous | Homozygous |
| | Deletion | Deletion | Deletion |
| PTEN | Single Copy | Single Copy | Single Copy |
| | Deletion | Deletion, Single | Deletion |
| | | Copy Mutation | |
| EGFR | Amplified | Amplified | — |
| | (variant 3) | (variant 3) | |
| TP53 | Mutant | — | — |
| Met. | Amplified | Amplified | Amplified |
| TERT Prom. | Mutant | Mutant | — |
| Others | — | Mutant MDM4 | PDGFR |
| | | & PIKC32B | Amplification, |
| | | Amplification | NF1 Loss, |
| | | | ATRX |
| | | | truncation |

IR Also Increases BCL-XL mRNA and Protein Expression in Human GBM Cells

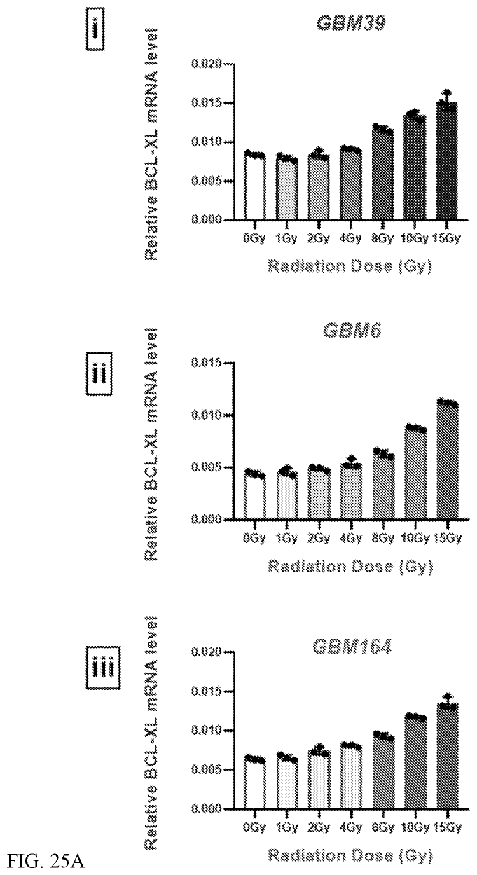

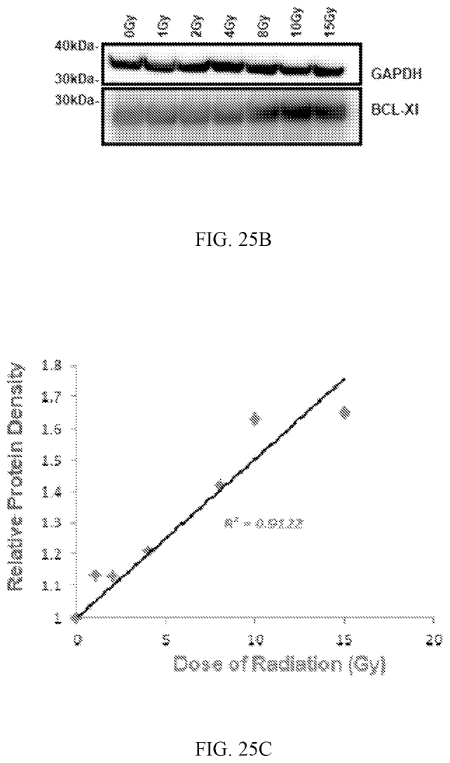

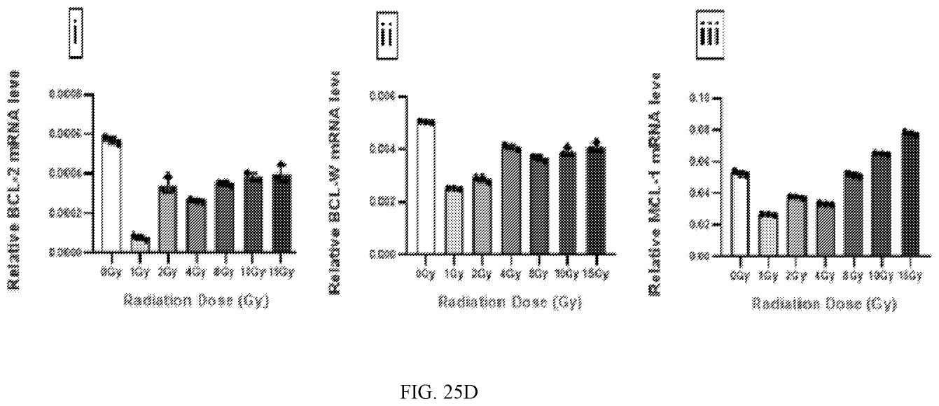

Cancer cells are notoriously resilient against cellular stresses such as DNA damage, hypoxia, nutrient deprivation and inflammation. B-cell lymphoma-extra large (BCL-XL) is an antiapoptotic protein embedded into organelle membranes, most importantly the outer mitochondrial membrane, where they can bind their BH3-only pro-apoptotic relatives, such as BID, BIM, and PUMA. Several lines of human GBM are selectively dependent upon BCL-XL after radiation (see, e.g., Rahman et al., bioRxiv doi: 10.1101/2020.06.03.132712 (2020)). In similar manner to PUMA, BCL-XL was also upregulated in response to radiation in a dose-dependent manner at 24 hours based on both mRNA (FIG. 25A) and protein (FIGS. 25B, 25C). For comparison, the transcriptional regulation of other anti-apoptotic BCL-2 family members BCL-2, BCL-W and MCL-1 was evaluated (FIG. 25D). Of these, only MCL-1 showed modest upregulation at highest doses of radiation (FIG. 25D.iii). These data suggest that BCL-XL may be mechanistically responsible for antagonizing the activity of PUMA after radiation to help maintain cell viability.

BCL-XL Interacts with PUMA More than any Other BH3-Only Family Member

To test if BCL-XL can sequester PUMA to prevent apoptosis, the extent to which PUMA or other pro-apoptotic BH3-only proteins may be present and sequestered as protein complexes with BCL-XL in non-radiated (0Gy) GBM cell lines was first examined.

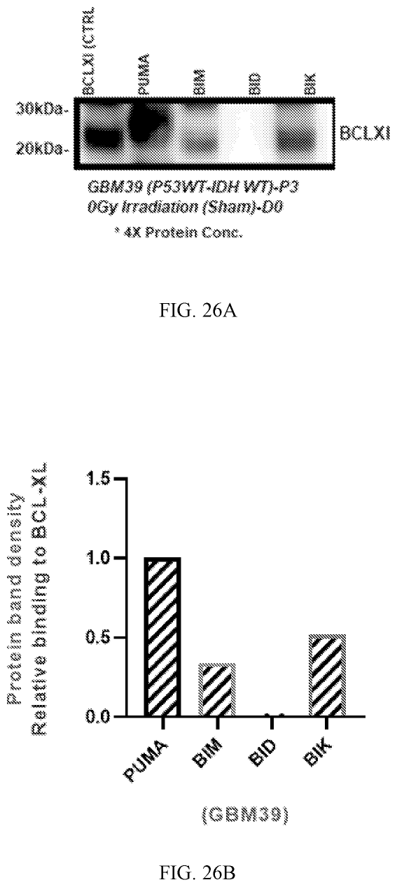

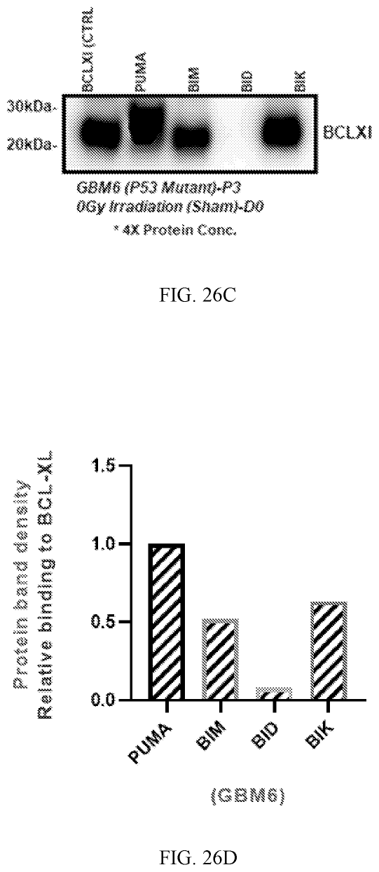

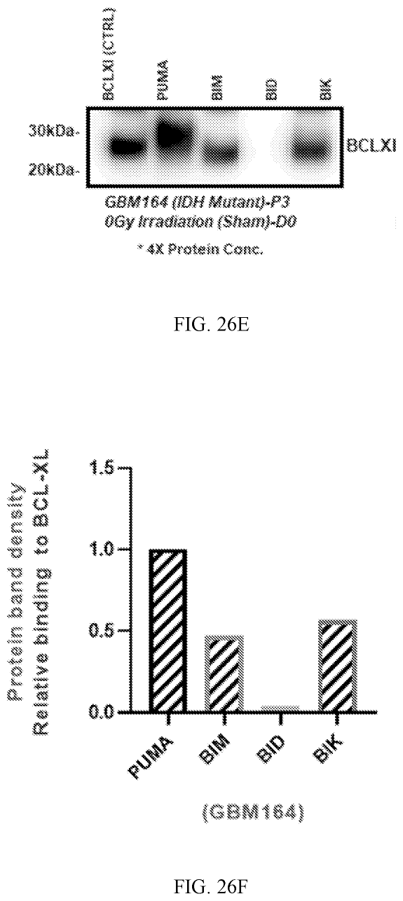

Given the relatively lower abundance of BCL-XL in non-radiated cells, 4 μg of protein lysate (“4×”) from each cell line was utilized to optimize evaluation of available protein complexes. Protein immunoprecipitation was performed using selective antibodies against the BH3-only pro-apoptotic proteins PUMA, BIM, BID and BIK. The relative level of BCL-XL present in each complex was quantified via co-IP for each cell line (FIG. 26). Pure BCL-XL was immunoprecipitated from the same lysate and included as a positive control (far left lane in FIGS. 26A, 26C, 26E). In each GBM cell line, a higher level of the BCL-XL protein in the protein complexes associated with PUMA was detected than any other BH3-only protein associated complexes (BIM, BID and BIK; FIG. 26). Densitometric quantification revealed that the amount of BCL-XL detected in the PUMA-associated complexes is about 40-50% higher than the amount detected in the second most upregulated BH3-only proteins in GBM cells, BIK, as shown in (FIG. 26B, D, F).

IR Increases the Interaction Between BCL-XL and PUMA More than Other BH3-BCL-XL Interactions

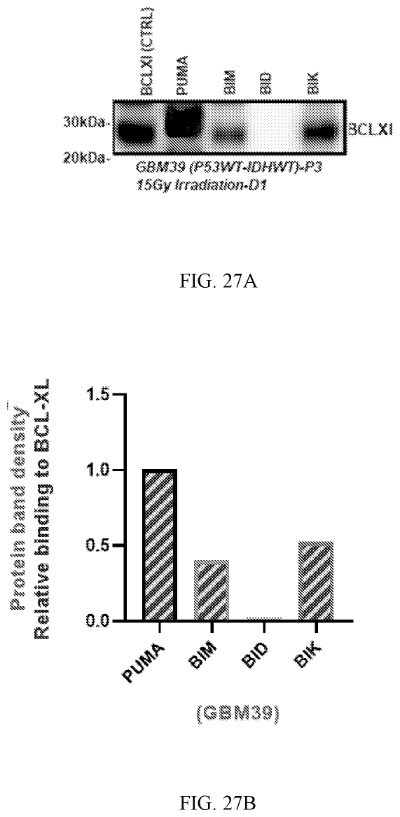

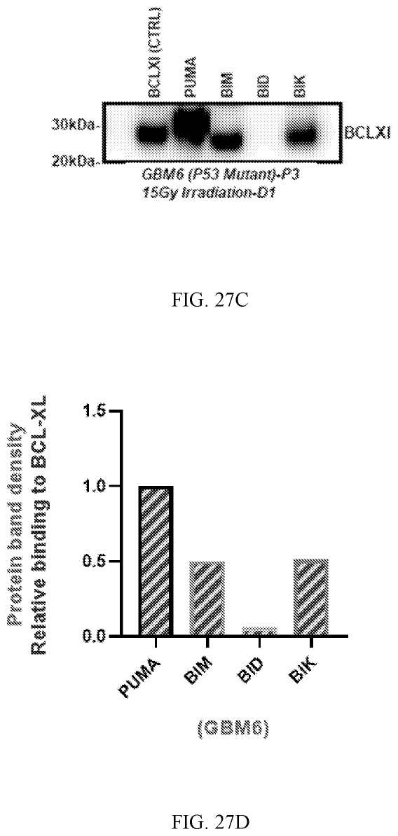

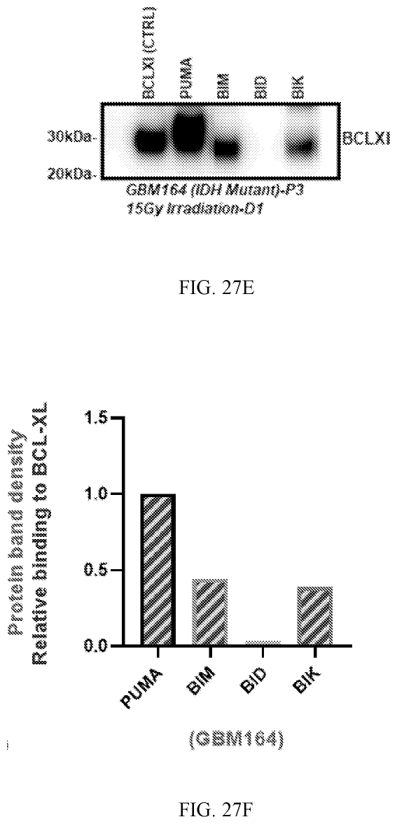

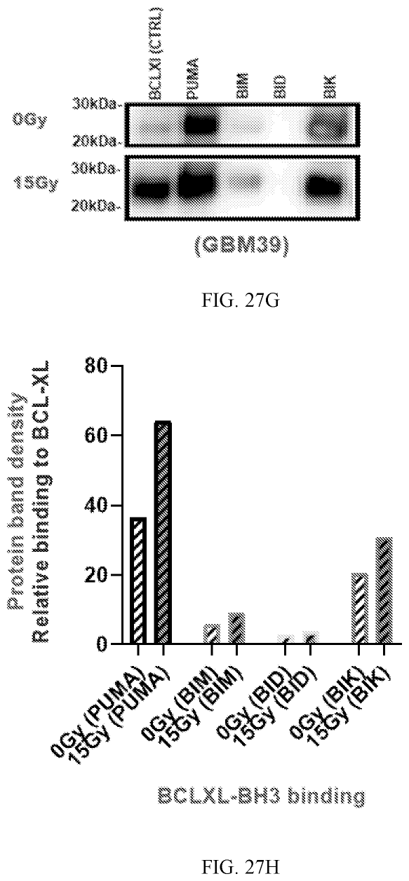

Since the BCL-XL-PUMA complex is relatively abundant in GBM cells (FIG. 26), and since BCL-XL and PUMA are both upregulated following radiation (FIGS. 24, 25), it was next examiner how radiation impacts the interaction of BCL-XL with PUMA relative to other pro-apoptotic BH3-only members (PUMA, BIM, BID, and BIK) isolated from each GBM cell line (GBM6, 39, and 164). For these studies, 1 μg protein lysate was used to account for the higher levels of BCL-XL and PUMA after radiation. In similar manner to the 0Gy state, 24 hours after 15Gy radiation, BCL-XL continued to be more abundant in the protein complexes associated with PUMA than any other BH3-only protein-associated complexes in each cell line (FIGS. 27A-27F). To specifically quantify the impact of radiation on the relative abundance of these BH3-BCL-XL complexes, bindings of BH-3 proteins to BCL-XL were examined by running equal concentrations of these immunoprecipitated samples from GBM39 (0Gy vs. 15Gy; FIG. 27G). Densitometric analysis revealed a marked increase in PUMA to BCL-XL, as compared to the minimum increase for BCL-XL binding to BIM, BID and BIK. These data are consistent with the idea that the radiation-induced increase in BCL-XL could serve to attenuate multiple pro-apoptotic BH-3 proteins, of which PUMA may be of particular importance (FIG. 27H).

PUMA Preferentially Interacts with BCL-XL and BCL-W

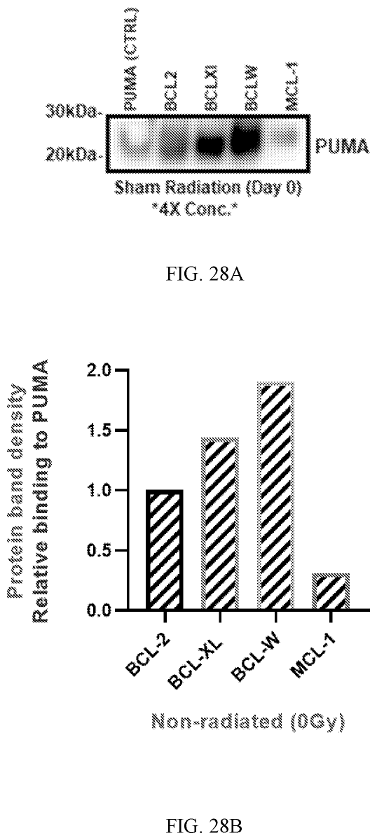

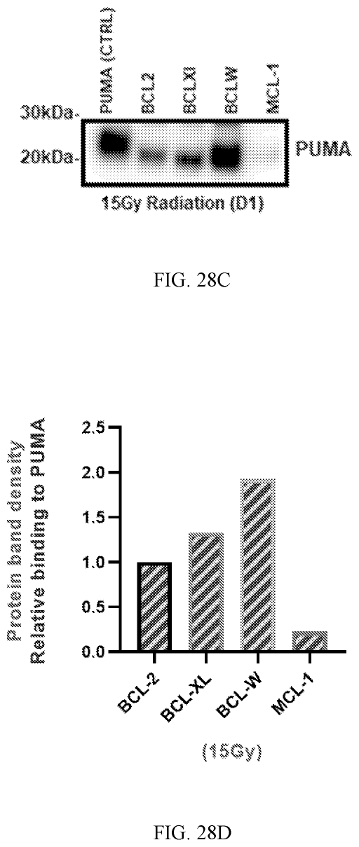

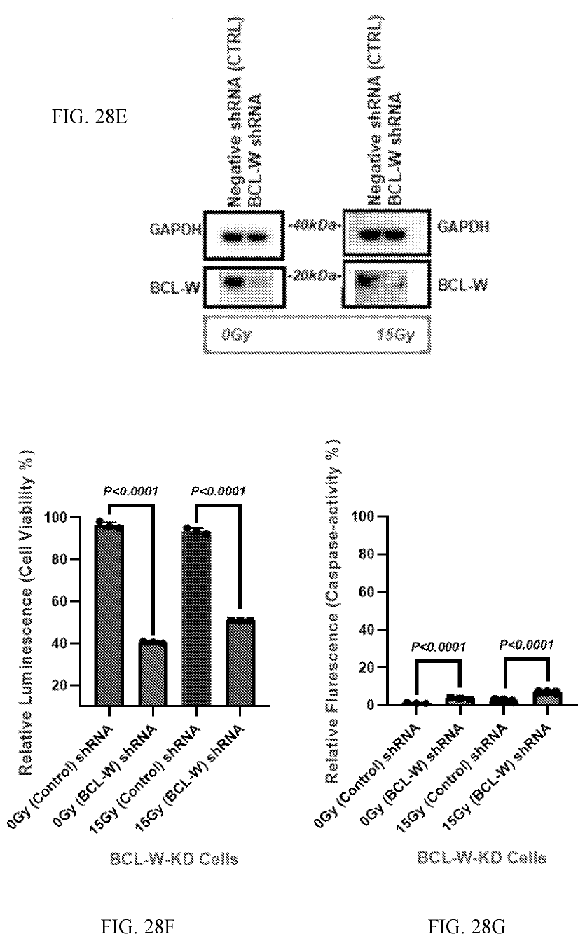

BCL-2 family proteins interact with each other to regulate cell fate. To determine the relative binding behaviors of BCL2 family anti-apoptotic members within GBM before and after radiation, immunoprecipitation (IP) and co-immunoprecipitation (Co-IP) were utilized to isolate protein complexes associated with BCL-2, BCL-XL, BCL-W, and MCL-1 from GBM39 cells 24 hours after 0Gy and 15Gy radiation (FIGS. 28A-28D). It was examined whether PUMA may appear in these complexes using the immunoprecipitated PUMA from the same lysates a positive control (left lane in FIG. 28A and FIG. 28C). Since relatively little PUMA protein is present in non-radiated GBM (FIG. 24), 4 μg (“4× conc”) of the protein lysate was used to obtain clear bands from non-radiated (0Gy) cells (FIG. 28A). 1 μg was utilized as in irradiated cells to avoid oversaturation (FIG. 28C). In both radiated and non-radiated cells, the highest PUMA protein content was found complexed with the antiapoptotic protein BCL-W, followed by BCL-XL then BCL-2. The lowest amount of PUMA protein was detected in the complexes associated with the antiapoptotic MCL-1 protein.

BCL-W does not Contribute to GBM Radio-Resistance.

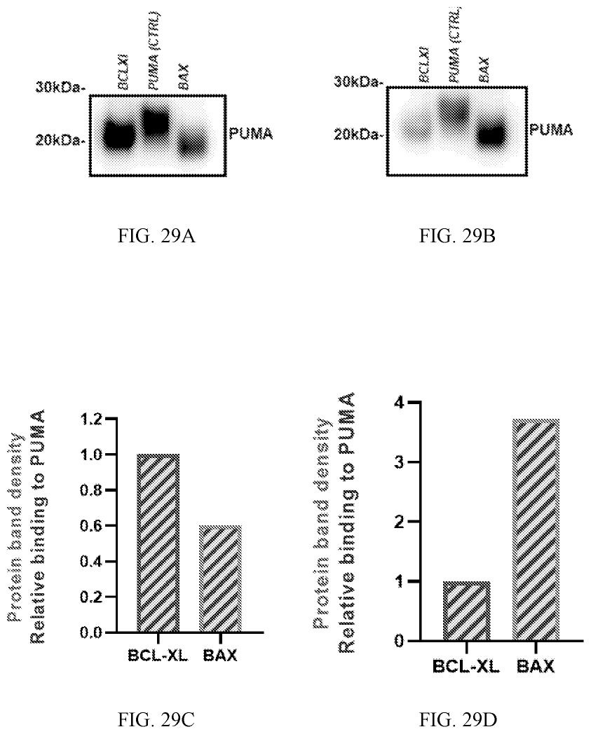

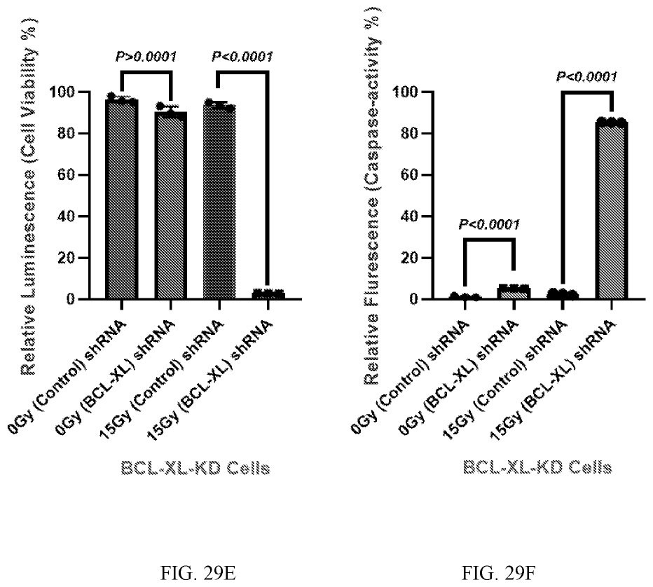

Since PUMA positively regulates glioma apoptosis and was found heavily complexed to the antiapoptotic protein BCL-W, it was tested if BCL-W knock-down could decrease glioma radio resistance. Using cellular ATP content to estimate cell viability (cell-titer glow), BCL-W shRNA knockdown decreased GBM viability to 40.56% (P<0.0001) of baseline levels in non-radiated (0Gy) cells and 50.86% (P<0.0001) in radiated (15Gy) cells (FIGS. 28E-28F). To determine if cell viability decreased due to apoptosis, caspase 3 activity was quantified and normalized to TRAIL-exposed cells wherein apoptosis occurs in 100% of cells. Despite over 50% decrease in cell viability in both radiated and non-radiated cells with BCL-W knockdown, relative caspase 3 activity was <10% in both groups (FIG. 28G). These results contrasted with those subsequently observed with BCL-XL knockdown (FIGS. 29E-29F) wherein relative caspase 3 activity in radiated cells exceeded 80%. These data suggest that BCL-W prevents both radiated and non-radiated GBM cells from undergoing non-apoptotic cell death but did not selectively implicate BCL-W in GBM radio-resistance.

PUMA Binds to BAX after BCL-XL Knock-Down Leading to Apoptotic Cell Death in GBM Cells

The findings above suggest a prominent role of PUMA in the BCL-XL dependency of multiple GBM cell lines after radiation. It was next asked whether PUMA may be facilitating or contributing to apoptosis. To address this question, and since PUMA is more available in irradiated cells than in non-irradiated cells, BCL-XL- and BAX-associated protein complexes were isolated from irradiated GBM39 either treated with a lentiviral vector with shRNA scrambled control (FIG. 29A) or shRNA to knock-down BCL-XL (FIG. 29B).

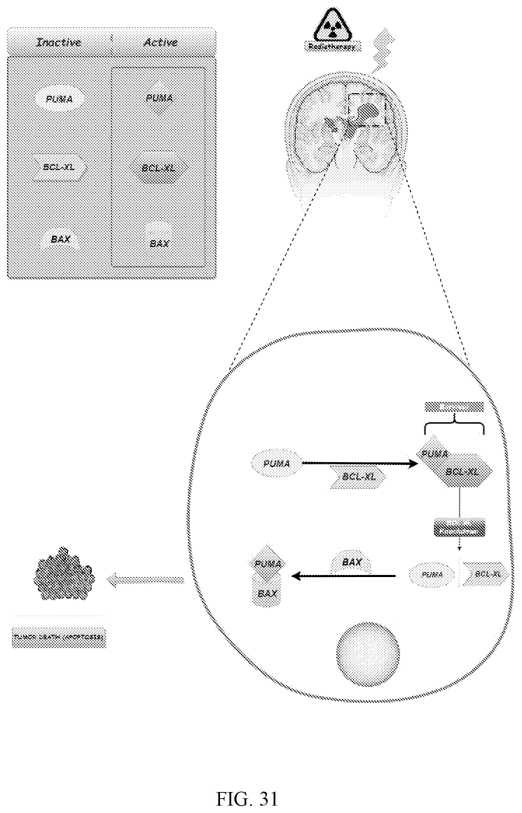

These isolated complexes were then examined for PUMA using a specific monoclonal antibody along with a purely isolated PUMA protein from these cells as a positive control, middle lane (FIGS. 29A, 29B). In cells treated with the scrambled shRNA, PUMA was found preferentially associated with BCL-XL. A relatively smaller amount of PUMA was found complexed to BAX (FIGS. 29A, 29C). When cells were treated with shRNA targeting BCL-XL, the amount of PUMA bound to BCL-XL was relatively modest (FIGS. 29B, 29D). The relative amount of PUMA bound to BAX was markedly higher. Collectively, these findings are consistent with a model wherein decreased PUMA sequestration upon BCL-XL knockdown leads to increased association with BAX, facilitating its activation to promote mitochondrial pore formation and apoptosis (FIG. 31).

### BAX and PUMA are Critical to Inducing Apoptosis in GBM Cells in Response to BCL-XL Knock-Down

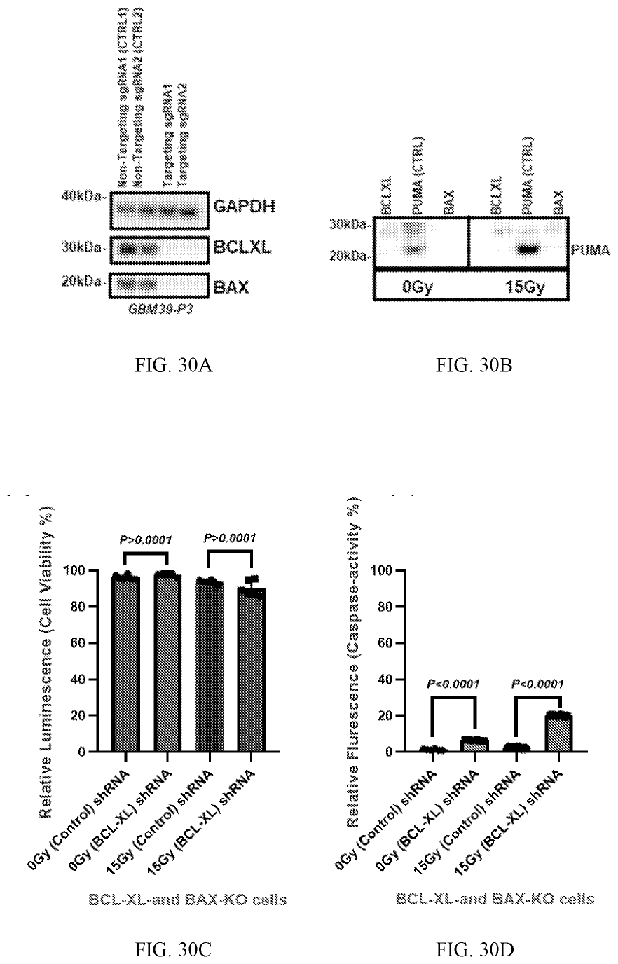

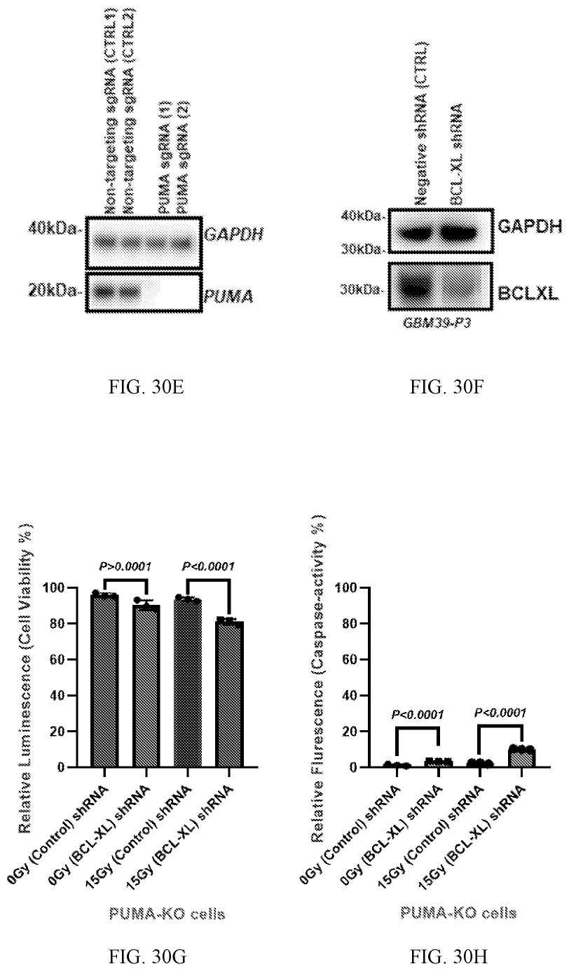

To determine if PUMA is both necessary and sufficient to induce apoptosis in the absence of BCL-XL, both BCL-XL and BAX were knocked-out from GBM39 cells (FIG. 30A). Since simultaneous CRISPR knockout of multiple lentiviral constructs adversely impacts cell viability and increases the chances of the off-target effect to happen, nanovesicles called gesicles were designed using the CRISPR/Cas9 Gesicle production system to simultaneously deliver synthetic guide RNAs (sgRNAs) targeting BCL-XL and BAX for concurrent knock out of both genes. Success of the knock-out by both western blot (FIG. 30A) and further confirming the absence of PUMA in BCL-XL- and BAX-associated protein complexes with (15Gy) and without (0Gy) radiation (FIG. 30B). Cell viability and caspase-3 activity were measured in these cells and found no significant difference in cell viability or caspase-3 activity in these knocked-out cells, as compared to compared to control (no knockout) cells with and without radiation (FIGS. 30C-30D). Since prior results revealed a profound induction of apoptosis with BCL-XL knockdown (FIGS. 29E-29F), these data suggested a critical role of BAX in apoptotic cell death in response to BCL-XL loss. A single gene PUMA knock-out was next established in GBM39 cells (FIG. 30E), using CRISPR/Cas9 technology, and by treating these cells with a lentiviral vector encodes for shRNA to knock-down BCL-XL (FIG. 30F), the change observed either in their cellular viability or caspase-3 activity was not of any significance from the cells treated with a scrambled shRNA as a control (FIGS. 30G-30H), suggesting a critical role of PUMA in apoptotic cell death in response to BCL-XL knock-down or knock-out the same as was observed regarding BAX (FIGS. 30A-30D).

### Example 7: Treating CNS Cancers

A human having been treated for a prior CNS cancer (e.g., a glioma such as a GBM) is administered one or more inhibitors of Bcl-xL polypeptide expression or activity to delay or prevent the development of a recurrent CNS cancer.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a central nervous system (CNS) cancer, wherein said mammal has been previously treated for a CNS cancer, wherein said method comprises:
administering an inhibitor of a B-cell lymphoma-extra large (Bcl-xL) polypeptide to said mammal; and
administering a chemotherapeutic agent to said mammal.

2. The method of claim 1, wherein said method comprises identifying said mammal as having said CNS cancer.

3. The method of claim 1, wherein said mammal is a human.

4-6. (canceled)

7. The method of claim 16, wherein said CNS cancer is selected from the group consisting of a brain stem glioma, a glioblastoma, an astrocytoma, an oligodendroglioma, an oligoastrocytoma, an ependymoma, a medulloblastoma, and a meningioma.

8. The method of claim 1, wherein said inhibitor of said Bcl-xL polypeptide is an inhibitor of Bcl-xL polypeptide activity.

9. The method of claim 8, wherein said inhibitor of said Bcl-xL polypeptide activity is selected from the group consisting of navitoclax, A1331852, A1155463, and WEHI-539.

10-11. (canceled)

12. The method of claim 1, wherein said chemotherapeutic agent is selected from the group consisting of temozolomide, selinexor, APO866, AMG-232, RG7388, and GMX1778.

13. (canceled)

14. The method of claim 13, wherein said inhibitor of said Bcl-xL polypeptide and said chemotherapeutic agent are administered in a single composition.

15. The method of claim 1, wherein said inhibitor of said Bcl-xL polypeptide and said chemotherapeutic agent are administered separately.

16. (canceled)

17. The method of claim 1, wherein said mammal, prior to said administering said inhibitor of said Bcl-xL polypeptide and said administering said chemotherapeutic agent, has not been treated for said CNS cancer for at least 1 month.

18. (canceled)

19. A method for treating a mammal having a recurrent CNS cancer, wherein said method comprises:
administering an inhibitor of a B-cell lymphoma-extra large (Bcl-xL) polypeptide to said mammal; and
administering a chemotherapeutic agent to said mammal.

20. The method of claim 19, wherein said method comprises identifying said mammal as having said recurrent CNS cancer.

21. The method of claim 19, wherein said mammal is a human.

22. (canceled)

23. The method of claim 19, wherein said mammal has, prior to said administration of said inhibitor of said Bcl-xL polypeptide, been treated for said CNS cancer with a radiation treatment.

24. The method of claim 19, wherein said mammal has, prior to said administration of said inhibitor of said Bcl-xL polypeptide, been treated for said CNS cancer with a chemotherapy treatment.

25. The method of claim 19, wherein said CNS cancer is selected from the group consisting of a brain stem glioma, a glioblastoma, an astrocytoma, an oligodendroglioma, an oligoastrocytoma, an ependymoma, a medulloblastoma, and a meningioma.

26. The method of claim 19, wherein said inhibitor of said Bcl-xL polypeptide is an inhibitor of Bcl-xL polypeptide activity.

27. The method of claim 26, wherein said inhibitor of said Bcl-xL polypeptide activity is selected from the group consisting of navitoclax, A1331852, A1155463, and WEHI-539.

28-29. (canceled)

30. The method of claim 19, wherein said chemotherapeutic agent is selected from the group consisting of temozolomide, selinexor, APO866, AMG-232, RG7388, and GMX1778.

31-32. (canceled)

33. The method of claim 19, wherein said inhibitor of said Bcl-xL polypeptide and said chemotherapeutic agent are administered separately.

34. (canceled)
